(19) United States
(12) Patent Application Publication
(10) Pub. No.: US 2026/0094673 A1
(43) Pub. Date: Apr. 2, 2026

(54) VIRUS SUBTYPING USING K-MER -BASED HASHING TECHNIQUES

(71) Applicant: Laboratory Corporation of America Holdings, Burlington, NC (US)

(72) Inventors: Qimin Zhang, Pittsburgh, PA (US); Kuncheng Song, Raleigh, NC (US); Hayden Nathaniel Brochu, Hillsboro, OR (US); Johnny Lai, Burlington, NC (US); Jonathan Toma, San Francisco, CA (US); Christos Petropoulos, Half Moon Bay, CA (US); Lakshmanan Krishnan lyer, North Attleboro, MA (US)

(21) Appl. No.: 19/346,049

(22) Filed: Sep. 30, 2025

Related U.S. Application Data
(60) Provisional application No. 63/701,423, filed on Sep. 30, 2024.

(51) Int. Cl. G16B 30/10; G16B 35/20

(52) U.S. Cl. CPC G16B 30/10; G16B 35/20

(57) ABSTRACT

The present disclosure relates to techniques for virus subtyping using k-mer hashing. Reference sets of hashed k-mers are obtained, each corresponding to a virus strain of a plurality of subtypes, with each reference set generated by applying a hash function to k-mers and storing each hashed k-mer as a digital key or index. One or more query sequences comprising viral nucleotide sequence data from a test sample are obtained, and for each query sequence, a test set of hashed k-mers is generated using the same hash function. Each test set is compared to each reference set to produce a k-mer matching score for each reference set. A query identity for the test sample is determined with respect to each virus strain based on the comparison. An assigned subtype for the test sample is determined based on the k-mer matching scores and/or the query identities using one or more decision rules.

### CROSS-REFERENCES TO RELATED APPLICATION

The present application claims priority and benefit from U.S. Provisional Application No. 63/701,423, filed Sep. 30, 2024, the entire contents of which are incorporated herein by reference for all purposes.

### FIELD

The present disclosure relates to techniques for analyzing biological sequence data, and in particular to techniques for viral sequence classification and subtyping using k-mer hashing and digital reference databases.

### BACKGROUND

Virus subtyping is in the technical field of virology and molecular diagnostics that classifies viruses into categories below the species level using genetic, antigenic, or phenotypic markers, typically through computational genomics that assigns a biological sample to a subtype based on its sequence content. Subtype information supports clinical decision making, drug resistance analyses, public health surveillance, and molecular epidemiology by resolving fine scale genetic structure that correlates with phenotypes and transmission dynamics. In practice, laboratories may generate data from whole genomes or targeted regions, and may work with raw reads or assembled consensus sequences produced by a variety of wet lab and bioinformatics workflows.

Accurate subtype determination is challenged by extensive genetic diversity, rapid mutation rates, and frequent recombination in many viral species, as exemplified by human immunodeficiency virus (HIV) and hepatitis C virus (HCV). In HIV, group M (a major lineage of HIV-1) comprises numerous subtypes and circulating recombinant forms, and frequent recombination generates mosaic genomes in which pol, env, and gag segments can trace to different subtype ancestries; within-host evolution under immune and drug pressure produces quasi-species that introduce minority variants, so a small region or a consensus alone can mask clinically relevant minorities. In HCV, multiple genotypes and more than one hundred subtypes create fine scale distinctions, while commonly sequenced regions such as the 5′ untranslated region (5′ UTR) are often too conserved to resolve subtypes, and greater discriminatory regions like Core E1 or NS5B may be partially covered or of variable quality; mixed genotype infections and reported recombinants further distribute subtype specific signal across the genome. These factors mean that subtype informative patterns are dispersed rather than localized, and they can conflict across regions when genomes are mosaic. When only a subset of loci is captured, or when coverage is uneven and reads are short, local alignments to conserved segments can dominate the analysis and blur differences among closely related subtypes, complicating confident and high resolution classification.

Subtype analysis is further complicated by data characteristics that vary across assays and platforms. Many workflows capture only portions of a viral genome, coverage can be uneven, and read length and error profiles differ by technology. Targeted amplification may introduce bias and dropouts, while contaminants and low complexity regions can confound similarity scoring. These factors can obscure subtype specific features, particularly when closely related subtypes differ through subtle, distributed patterns across the genome. Common comparison methods rely on database similarity or alignment, which can emphasize conserved segments and blur distinctions among neighboring subtypes while underrepresenting minority or recombinant signals. Position dependent alignments are sensitive to gaps, indels, and structural variation, and outcomes can hinge on heuristic parameters, especially when inputs are short reads or partial genomes.

Reliable subtyping depends on high quality reference collections that are comprehensive, current, and consistently labeled across subtypes and genotypes. Incomplete sequences, duplicates, mis-annotations, and inconsistent nomenclature reduce resolution and can propagate systemic errors, so effective governance includes clear curation criteria, versioning and update cadence, and traceability to support reproducibility and audit. Operational requirements in clinical and production settings include high throughput, short turnaround time, bounded memory and compute, and end to end automation. Outputs must be interpretable, with transparent confidence levels and explicit undetermined results when evidence is insufficient. Methods that generalize across viruses and remain effective on different genomic regions are valued as assays evolve and new strains emerge, and validation and reporting benefit from reproducible software environments and outputs that integrate with laboratory information systems and downstream clinical reporting.

### BRIEF SUMMARY

In various embodiments, a computer-implemented method is provided, comprising: obtaining a plurality of reference sets of hashed k-mers, wherein each reference set of the plurality of reference sets corresponds to a virus strain of a plurality of subtypes of a virus, wherein each reference set is generated by performing a hash function on k-mers of the virus strain, and wherein each hashed k-mer is stored as a digital key or index to store corresponding k-mer; obtaining one or more query sequences for a test sample, wherein the query sequence comprises viral nucleotide sequence data derived from the test sample; generating, for each of the one or more query sequences, a test set of hashed k-mers for the test sample using the hash function, thereby generating one or more test sets; comparing the one or more test sets to each reference set of the plurality of reference sets to generate a k-mer matching score for each reference set; determining a query identity for the test sample with respect to each virus strain based on the comparison; and determining an assigned subtype for the test sample based on the k-mer matching scores and/or the query identities using one or more decision rules.

In some embodiments, each reference set and each test set is stored in a data structure of a specific type, and wherein the data structure of the specific type is a hash table or a hash set.

In some embodiments, the k-mer matching score is generated based on a number or percentage of hashed k-mers from the test set that are present in the reference set.

In some embodiments, the obtaining the plurality of reference sets of hashed k-mers comprises: accessing a virus data store to obtain a plurality of virus strains of the plurality of subtypes of the virus, wherein each virus strain of the plurality of virus strains has a genome sequence and an associated subtype annotation; generating, for each virus strain of the plurality of virus strains, a set of k-mers by extracting nucleotide sequences of a specific length from the genome sequence using a sliding window; and performing, for each virus strain of the plurality of virus strains, the hash function on each k-mer of the set of k-mers to generate a digital representation of the hashed k-mer, thereby obtaining the set of hashed k-mers for the virus strain.

In some embodiments, the specific length is 15.

In some embodiments, the performing comprises: assigning an integer value to each k-mer of the set of k-mers; combining the integer values based on the hashing function to generate a unique or nearly unique integer for each k-mer; and assigning each resulting integer as the digital key or index in a hash table or a hash set.

In some embodiments, the generating the test set comprises: generating a set of k-mers for the test sample by extracting nucleotide sequences of the specified length from the query sequence; and performing the hashing function on each k-mer of the set of k-mers to generate a digital representation of the hashed k-mer, thereby generating the test set of hashed k-mers.

In some embodiments, the query identity is calculated as a percentage of bases in the genome sequence of the virus strain covered by matching k-mers from the test set.

In some embodiments, the obtaining the one or more query sequences comprises obtaining nucleic acids from the test sample, sequencing the nucleic acids using a high-throughput sequencing technique or capillary electrophoresis sequencing, and generating raw reads or consensus sequences.

In some embodiments, the test sample is a biological sample or an environmental sample.

In some embodiments, the k-mer matching score for the reference set is computed by assigning a positive weight to each matching k-mer and a penalty score to each unmatched k-mer.

In some embodiments, the positive weight is equal to a length of the k-mer.

In some embodiments, the determining the assigned subtype comprises: filtering virus strains with a query identity below a predetermined threshold; and determining the assigned subtype based on remaining virus strains, wherein when candidate strains meeting a predetermined criterion of the query identity share a same subtype, assigning the subtype to the test sample, and when the candidate strains meeting the predetermined criterion of the query identity do not all share the same subtype, applying a weighted voting rule to assign a first subtype and a second subtype based on the weighted voting rule, wherein the first subtype is assigned a higher confidence score than a confidence score of the second subtype.

In some embodiments, the computer-implemented method further comprises updating the virus data store and/or the reference sets based on a predetermined schedule.

In some embodiments, the virus is human immunodeficiency virus (HIV), hepatitis B virus (HBV), hepatitis C virus (HCV), or hepatitis delta virus (HDV).

In some embodiments, the determining the query identity comprises: identifying a subset of k-mers from the test sample that have matching hashed values in the reference set for the virus strain; generating a consensus sequence for the test sample with respect to the virus strain based on the subset of k-mers; and calculating the query identity as a percentage of bases in a genome sequence of the virus strain that are covered by the consensus sequence.

In some embodiments, the consensus sequence is generated by, at each nucleotide position in the genome sequence, selecting a consensus base based on a most frequently represented nucleotide by overlapping the k-mers in the subset that cover that position.

In some embodiments, the assigned subtype for the test sample is (i) a subtype from the plurality of subtypes or (ii) marked as “undetermined.”

In some embodiments, a system is provided that includes one or more processors and a non-transitory computer readable medium containing instructions which, when executed on the one or more processors, cause the one or more processors to perform part or all actions or operations in one or more methods or processes disclosed herein.

In some embodiments, a non-transitory computer readable storage medium is provided comprising computer program instruction that, when executed by one or more processors, cause the one or more processors to perform part or all actions or operations in one or more methods or processes disclosed herein.

In some embodiments, a computer-program product is provided that is tangibly embodied in a non-transitory machine-readable medium and that includes instructions configured to cause one or more data processors to perform part or all actions or operations in one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope described. Thus, it should be understood that although the present subject matter has been specifically disclosed by embodiments and optional features, modification and variation of the concepts herein disclosed may be resorted to by those skilled in the art, and that such modifications and variations are considered to be consistent with the principles described and the scope set forth in the claims.

### BRIEF DESCRIPTION OF THE DRAWINGS

The drawings illustrate certain embodiments of the technology and are not limiting. For clarity and ease of illustration, the drawings are not made to scale and, in some instances, various aspects may be shown exaggerated or enlarged to facilitate an understanding of particular embodiments. The disclosed techniques will be better understood in view of the following non-limiting figures, in which:

FIG. 1A illustrates the human immunodeficiency virus (HIV) genome architecture in accordance with various embodiments.

FIG. 1B illustrates the hepatitis C virus (HCV) genome architecture in accordance with various embodiments.

FIG. 2 shows an exemplary environment for performing virus subtyping using k-mer hashing techniques in accordance with various embodiments.

FIG. 3 illustrates an exemplary workflow for a k-mer-based virus subtyping techniques in accordance with various embodiments.

FIG. 4 illustrates a high-level schematic workflow for virus subtyping using a k-mer hashing approach in accordance with various embodiments.

FIG. 5 is a flowchart illustrating a process for performing virus subtyping using a position-independent k-mer hashing approach in accordance with various embodiments.

FIG. 6A shows k-mer typing results for non-B samples in accordance with various embodiments.

FIG. 6B shows a comparative table summarizing the performance evaluation of the k-mer-based HIV subtyping pipeline across multiple test samples in accordance with various embodiments.

FIG. 6C is a table illustrating a concordance analysis of 2,369 HIV samples in accordance with various embodiments.

FIG. 6D illustrates a data curation process in a HDV genotyping pipeline in accordance with various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

The ensuing description provides examples of preferred embodiments only and is not intended to define the scope, applicability, or configuration of the subject matter described. Rather, the ensuing description of the preferred exemplary embodiments will provide those skilled in the art with sufficient detail for implementing various embodiments. It is recognized that various changes may be made in the function and arrangement of elements without departing from the principles described and the scope set forth in the claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

### I. INTRODUCTION

Virus subtyping relates to computational sequence classification that maps genomic information (e.g., nucleotide data) from a biological sample to a predefined subtype taxonomy, for example, by quantifying sequence-content similarity against curated reference genomes and subtype labels. Subtyping in practice is carried out on varied inputs, including FASTA consensus sequences and FASTQ reads, and is usually used under conditions where only portions of a genome are sequenced and where coverage and read quality differ by platform. Accurate subtyping informs clinical decision-making, epidemiological investigations, and research into viral evolution, drug resistance, and vaccine design. However, the process of subtyping viruses presents significant challenges due to the complexity and diversity of viral genomes, as well as technical and computational constraints.

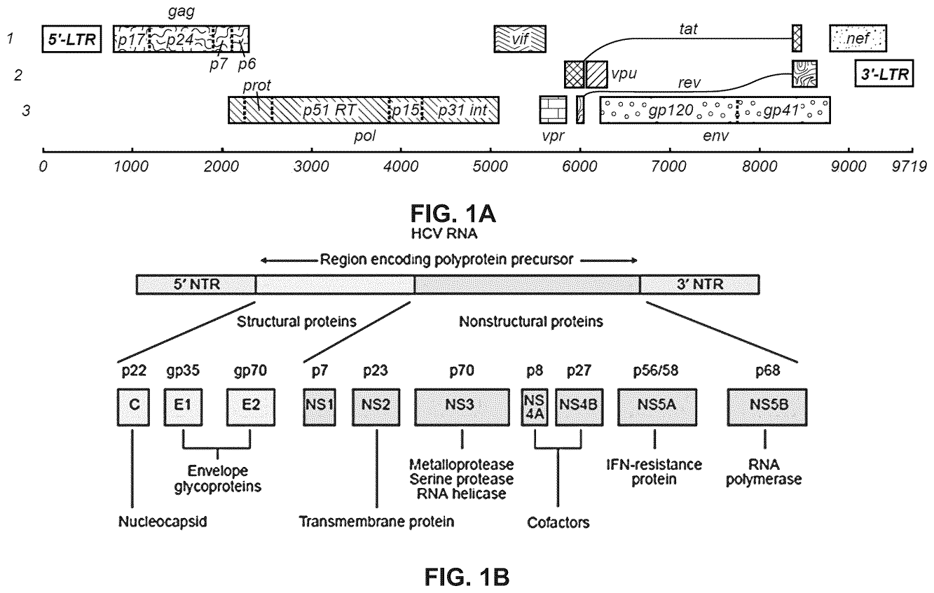

FIG. 1A illustrates the genomic structure of human immunodeficiency virus (HIV), which is an RNA virus with a genome approximately nine to ten kilobases in length. As shown in FIG. 1A, the HIV genome contains key structural genes, including gag, pol, and env, as well as accessory genes such as vif, vpr, vpu, tat, rev, and nef, and flanking long terminal repeats on both ends. In most routine diagnostic and surveillance workflows, subtyping assays focus on limited genomic regions such as pol or env. However, the propensity of HIV to undergo recombination can generate mosaic genomes, where the gag, pol, and env regions each originate from different subtype ancestries. This dispersal of subtype-informative signals throughout the genome makes it difficult to confidently classify subtypes when only a subset of loci or short amplicons are available. The issue becomes even more pronounced when minority viral populations within a host, known as quasi-species, are present, as these can be hidden in consensus sequences and evade detection.

FIG. 1B illustrates the genomic structure of the Hepatitis C Virus (HCV), which includes a 5′ untranslated region (5′ UTR), structural proteins such as C, E1, and E2, and nonstructural proteins including NS3, NS4A, NS4B, NS5A, and NS5B. Different regions of the HCV genome have varying abilities to discriminate among subtypes. For instance, the 5′ UTR is highly conserved and usually insufficient to resolve subtypes, while regions such as Core E1 or NS5B contain more subtype-specific information. These informative regions are commonly sequenced as short amplicons, typically a few hundred bases in length, which may only partially cover the region of interest or may be compromised by variable sequence quality. The challenge is further compounded by mixed genotype infections and the occurrence of recombinants, which distribute subtype-specific signals across the genome. When only short reads or partial genomes are available, signals from minority populations or recombination breakpoints can be diluted, leading to difficulty in achieving high-resolution classification.

From a computational perspective, conventional subtyping pipelines generally compare sample sequences to reference databases using local alignment algorithms such as Nucleotide Basic Local Alignment Search Tool (BLASTn), which compares one or more nucleotide query sequences to a subject nucleotide sequence or a database of nucleotide sequences and is routinely employed in clinical workflows. In typical deployments, per-query runtime scales approximately linearly with the database size, O(N), while exact dynamic-programming alignments on a single query of length n against a subject of length m have worst-case time complexity O(n*m); for Q queries against a database of size N, aggregate cost is O(QN), and memory requirements for indexing and search generally scale as O(N). For example, aligning an approximately 9 to 10 kb HIV genome against a large reference database of complete genomes requires scanning O(N) subjects per query and computing O(n*m) alignment scores per candidate, which in practice can be time consuming at production scale. Additionally, because local alignment tends to prioritize conserved genomic regions, it can blur distinctions among closely related subtypes and miss minority or recombinant signals. Results are position-dependent and sensitive to gaps, insertions, deletions, and other structural variation, and they can vary with user-defined parameters. When inputs are short reads or partial genomes, these constraints are amplified, increasing turnaround time and computational cost in high-throughput or clinical settings.

Recent developments in virus subtyping have explored the use of k-mers to improve classification accuracy and computational efficiency. In these approaches, k-mers are used for seeding and then refined with position-dependent alignments, for example seed extension with Needleman-Wunsch between seed extends, and multi-mapping resolution via ConClave scoring per template, which introduces runtime overhead and sensitivity to gaps and indels. Even when heuristic k-mer mapping is used to quickly narrow down candidate templates, the subsequent fine alignment stage for each query-template pair carries computational costs (e.g., O(n*m) complexity) that scale with both the length of the query and the size of the reference database. As reference databases become larger and more redundant, the memory and computational demands of these approaches increase, making production-scale subtyping more time consuming.

Additionally, these approaches usually assemble a reference-guided consensus sequence for each template, which means that supporting evidence for subtyping is aggregated along specific alignment paths, rather than across the entire genome, which also biases the results toward conserved regions. Consequently, signals from minority variants or recombinant genomes may be underrepresented, and these approaches fail to capture the full spectrum of genetic diversity present in the sample. In practical terms, these characteristics can reduce the resolution with which closely related viral lineages are distinguished and can contribute to misclassification or low-confidence outcomes. This is especially true when the input data consists of short reads, partial genomes, or mixtures of strains, as might be encountered in clinical or epidemiological settings. For example, when only a subset of the HIV genome (such as short pol amplicons) is sequenced, or when only a partial NS5B region of HCV is available, the sensitivity of position-dependent alignment methods is limited, and minority or recombinant signals may be masked or missed entirely.

To address the limitations and challenges associated with both conventional alignment-based subtyping and k-mer seeding methods, disclosed herein are techniques for performing k-mer hashing-based virus subtyping using a position-independent approach. In an illustrative embodiment, the disclosed approach includes accessing a virus database comprising virus strains of a plurality of subtypes (such as HIV, HCV, or others) and generating a reference set of hashed k-mers for each strain. One or more query sequences from a test sample are also obtained, and each query sequence is processed to generate a corresponding test set of hashed k-mers. The test set is compared to each reference set to compute a k-mer matching score and determine the query identity. Based on the k-mer matching score and/or the query identity, an assigned subtype is determined for the test sample.

The disclosed techniques enhance both the sensitivity and resolution of virus subtyping by enabling detection of informative signals that are distributed throughout the entire viral genome. By applying a position-independent k-mer hashing method, the approach is able to compare the complete set of k-mers from the input query sequence to multiple reference sets constructed from complete genomes within a practical timeframe. This genome-wide assessment ensures that all subtype-informative signals, whether they are located in conserved or variable regions, are considered in the analysis. As a result, the method supports accurate subtype classification even when only partial genome data or short reads are available. This improvement directly addresses the challenge of missed recombinant or minority signals that can arise when traditional methods focus only on targeted regions.

The present approach improves the ability to detect recombination events, quasi-species diversity, and mixed infections by using a position-independent k-mer hashing workflow. Unlike conventional position-dependent or consensus-based subtyping methods, which may mask or dilute signals from minority or recombinant variants when mosaic genomes or mixed populations are present, the disclosed techniques quantify similarity based on the aggregate presence of matching k-mers between the query and reference sets without considering their genomic locations. This enables more reliable detection of recombinants, minor variants, and mixed infections that can occur in clinical or epidemiological samples, and it improves the accuracy of subtyping in situations where conventional workflows may misclassify or overlook such cases.

The disclosed approaches achieve a substantial improvement in computational efficiency by reducing the time required for comparing k-mers between query and reference sets from O(n) to O(1) time. This can be accomplished by storing each reference set of hashed k-mers in a hash table, which allows for constant-time membership checks regardless of the size of the reference database. As a result, the disclosed workflow efficiently processes large and redundant reference databases, enabling rapid subtyping even as the number of strains and subtypes increases. This computational advance makes it feasible to perform scalable, high-throughput virus subtyping with practical turnaround times, even in production settings. The efficiency gain is particularly important for detecting informative signals across complex viral genomes, including those of HCV, Hepatitis Delta Virus (HDV), and HIV, where subtype-informative regions are distributed throughout the genome and rapid, genome-wide comparison can be critical for accurate classification

The disclosed approaches also offer flexibility and broader applicability compared to current subtyping tools. The disclosed approaches are not limited to particular genomic regions or virus types and can be adapted to a range of sequencing targets and viral species. Reference databases can also be updated as new strains and subtypes are validated and made available, ensuring that the subtyping workflow remains effective as viral diversity evolves. Beyond these examples of HIV, HCV, and HDV, the disclosed approaches can be readily adapted for subtyping other viruses such as influenza (where reassortment and rapid evolution present unique challenges), SARS-CoV-2 and other coronaviruses (where rapid identification of emerging variants is essential), human papillomavirus (HPV) with its many genotypes, flaviviruses like dengue and Zika, hepatitis B virus (HBV), enteroviruses such as poliovirus and coxsackievirus, norovirus, rotavirus, and respiratory syncytial virus (RSV). As additional viral reference data are generated, the subtyping workflow can be updated to include new targets, thereby supporting ongoing surveillance, outbreak response, and research across a wide range of clinically and epidemiologically important viruses.

### II. DEFINITION OF TERMS

As used herein, the articles “a” and “an” are used herein to refer to one or to more than one (i.e., at least one) of the grammatical object of the article. By way of example, an element means at least one element and can include more than one element.

As used herein, “and/or” refers to and encompasses any and all possible combinations of one or more of the associated listed items, as well as the lack of combinations where interpreted in the alternative (“or”).

As used herein, when an action is “based on” something, this means the action can be based at least in part on at least a part of the something.

As used herein, the terms “comprising,” “including,” or “having,” and variations thereof, are intended to indicate that the elements listed thereafter, as well as equivalents and additional elements, are encompassed. Embodiments recited as “comprising,” “including,” or “having” certain elements are also contemplated as covering embodiments “consisting essentially of” or “consisting of” those certain elements.

As used herein, the term “confidently” or “confidence” refers to the degree of certainty with which an analytical method or algorithm assigns a subtype or classification to a viral sequence, based on predefined statistical thresholds, scoring criteria, or quality metrics established by the subtyping workflow or reference database. An assignment is considered to be made “confidently” if the relevant metric (e.g., a k-mer matching score, query identity, probability, or other quantitative measure) meets or exceeds a specified threshold for accuracy or reliability, such that the risk of misclassification is acceptably low according to the standards of the field or the parameters set by the workflow. For example, in digital subtyping workflows, a minimum confidence score may be set (e.g., 0.8 or eighty percent), so that a viral sequence is only assigned to a particular subtype if the associated metric reaches or surpasses this threshold. Assignments falling below the threshold may be reported as ambiguous, at a higher-level classification, or left unassigned. In some embodiments, the minimum confidence threshold can be determined empirically by evaluating performance on control samples or benchmark datasets and selecting a cutoff that balances sensitivity and specificity. Alternatively, the threshold may be set by the user, or determined according to published best practices, guidelines in the field, or recommendations from developers of the subtyping tool or reference database.

As used herein, the term “consensus sequence” refers to a nucleotide sequence that is generated by assembling and combining multiple sequencing reads or fragments derived from a single sample, in such a way that, at each position in the sequence, the most frequently observed nucleotide is assigned. The consensus sequence represents the predominant genetic composition of the viral population within the sample and is typically used for downstream analyses such as subtyping. Consensus sequences may be generated from raw sequencing data in FASTQ format or may be provided directly in FASTA format as an input for subtype classification.

As used herein, the term “data store” refers to a location, system, or technology used for the retention and safekeeping of data, which includes, but is not limited to, file systems, cloud storage services, object stores, databases, digital repositories, tape archives, and other media or mechanisms capable of holding data in any structured or unstructured form. As used herein, the term “digital repository” refers to a managed digital system or platform specifically designed for the curation, preservation, and controlled dissemination of digital content, which is typically accompanied by metadata, access controls, and long-term integrity features. As used herein, the term “database” refers to an organized collection of structured data that is electronically stored and managed by a database management system (DBMS). Databases may be relational (SQL) or non-relational (NoSQL), and are optimized for transactional consistency, data integrity, and complex operations. In some embodiments, the terms “data store,” “digital repository,” and “database” may be used interchangeably.

As used herein, “hashing” refers to transforming a data element, such as a nucleotide sequence, a k-mer, or any other string or record, into a deterministic digital digest or hash value by applying a hash function. While collisions are possible for fixed-length digests, well-chosen hash functions make them computationally infeasible for practical purposes. Hashing supports fast indexing, deduplication, integrity checks, k-mer sketching, and efficient comparisons across large datasets. As used herein, the terms “hash function” and “hashing function” are used interchangeably, and examples of hashing functions include cryptographic hash functions such as SHA-256, SHA-3-256, SHA-1, MD5, BLAKE2, and BLAKE3, and non-cryptographic hash functions including MurmurHash3, xxHash, and CRC32.

As used herein, a “k-mer” refers to a contiguous subsequence of length k nucleotides extracted from a longer nucleotide sequence, such as a read, contig, or genome. For example, given a nucleotide sequence “AGCTTAGC,” the 3-mers are “AGC,” “GCT,” “CTT,” “TTA,” “TAG,” and “AGC.” The value of k can be any positive integer, with the choice of k depending on the application and desired balance between specificity and sensitivity. As used herein, a “hashed k-mer” refers to a digital representation of a k-mer generated by applying a hash function to the nucleotide string of the k-mer. The hash function converts the k-mer into a unique or nearly unique numerical or alphanumeric value, enabling efficient storage, comparison, and retrieval in computational workflows. Each hashed k-mer may be stored in a digital data structure, such as a hash table or set, which can be used to build reference databases for known viral strains or to represent the set of k-mers derived from a query sequence.

As used herein, the term “or” encompass both the inclusive sense, where either one or both of the conditions or elements can be present, and the exclusive sense, where only one of the conditions or elements can be present. In some instances, the term “or” is used interchangeably with “and/or.”

As used herein, the term “pipeline” refers to an automated and coordinated sequence of computational procedures or workflows that are implemented based on computer hardware and are designed to process input data such as viral sequencing reads or consensus sequences through a series of analytical steps for subtyping or classification. The pipeline may include, but is not limited to, modules or routines for k-mer extraction, hashing, comparison of hashed k-mers to reference sets or reference bubbles, computation of similarity scores, and final subtype assignment. In some embodiments, the pipeline may be implemented using executable scripts, compiled software, microservices, or containerized environments operating on general-purpose processors, high-performance computing clusters, or cloud infrastructure. The pipeline is engineered to promote user-friendliness and automation, incorporating self-contained reference databases and allowing for the processing of both FASTA and FASTQ input formats with minimal manual intervention. As used herein, the terms “pipeline,” “workflow,” and “processing workflow” may be used interchangeably to describe the automated data processing and analysis system that enables scalable and reproducible virus subtyping in clinical, research, or surveillance settings.

As used herein, the term “query sequence” refers to a nucleotide sequence obtained from a test sample that is subject to analysis for the purpose of classification, such as subtyping. A query sequence may originate from raw sequencing data (for example, a single read or a collection of reads from FASTA or FASTQ files), an assembled consensus sequence, or any nucleotide fragment for which viral subtype or related classification is to be determined. The query sequence is compared against reference sequences or reference sets to assess similarity, assign subtype, or make other analytical determinations within the described workflow.

As used herein, “reads” (e.g., “a read,” “a sequence read”) are nucleotide sequences produced by sequencing processes known in the art. Reads can be generated from one end of nucleic acid fragments (“single-end reads”), and sometimes are generated from both ends of nucleic acid fragments (e.g., paired-end reads, double-end reads).

As used herein, the term “reference genome” or “reference genomic sequence” refers to any known, sequenced, or characterized genome, whether partial or complete, of an organism or virus, which is used for comparison or reference in analyzing sequences obtained from a sample. A “genome” refers to the total genetic information of an organism or virus, expressed as nucleic acid sequences. A reference genomic sequence may be an assembled or partially assembled sequence from one or more individuals or isolates. In some embodiments, a reference genome may include sequences assigned to chromosomes or genomic segments, and for viral genomes, may represent entire genomes, segments, or specific genes relevant for subtyping.

As used herein, the term “reference set” refers to a collection of digital representations, such as hashed k-mers, that are derived from a reference genome or reference genomic sequence. Each reference set serves as a digital fingerprint of a specific viral strain, subtype, or genomic segment, and is used for rapid comparison with query sequences in computational workflows. As used herein, the term “test set” refers to a collection of digital representations, such as hashed k-mers, that are generated from a query sequence obtained from a sample under analysis. The test set represents the k-mer content of the sample and is compared against one or more reference sets to assess similarity, assign subtype, or make other analytical determinations within the computational workflows.

As used herein, the term “reference bubble” refers to a data structure or set that contains all hashed k-mers generated from a particular reference genome or segment. Each reference bubble corresponds to one reference genome or strain and is constructed by extracting all possible k-mers of a specified length from the reference sequence, hashing each k-mer, and storing the resulting values in a digital data structure such as a hash table or set. Reference bubbles enable efficient, position-independent comparison between query sequences and large, redundant reference databases, supporting high-throughput subtyping workflows. In some embodiments, the terms “reference set” and “reference bubble” can be used interchangeably.

As used herein, the term “sample” refers to a biological or chemical material collected and processed for analysis in a laboratory, clinical, surveillance, or research setting. In the context of viral subtyping, a sample may include, but is not limited to, blood, plasma, serum, swabs, tissue, cell culture supernatants, or other materials containing viral nucleic acids. The sample can be processed to extract nucleic acids for sequencing or amplification, and may be derived from a subject, an environmental source, or a laboratory culture.

As used herein, the term “subject” refers to any individual organism, including humans and non-human animals, from which a sample may be collected for analysis. In some embodiments, a subject is a human subject.

As used herein, the term “subtype” refers to a predefined genetic classification within a given viral species, distinguishing groups of viruses that share a higher degree of sequence similarity with each other than with viruses in other subtypes. Subtypes are designated based on established taxonomic or phylogenetic criteria, such as those defined for HIV (e.g., subtypes A, A1, A2, A3, A4, A6, A7, A8, B, C, D, F1, F2, G, H, J, K, L, N, O, P, U, AE, AG, AB, DF, BC, CD, BF, BG, Complex, and SIV) or HCV (e.g., subtypes 1a, 1b, 2a, etc.). Assigning a subtype provides information relevant to epidemiology, clinical management, and research.

As used herein, the term “substantially” means approximately, nearly, or to a large extent, and includes the case of full or exact conformity, as would be understood by a person of ordinary skill in the art. As used herein, “approximately” or “about” means within [a percentage] of the stated value, illustratively±0.1 percent, ±1 percent, ±5 percent, ±10 percent, or ±20 percent, unless otherwise specified.

### III. EXEMPLARY VIRUS SUBTYPING ENVIRONMENT

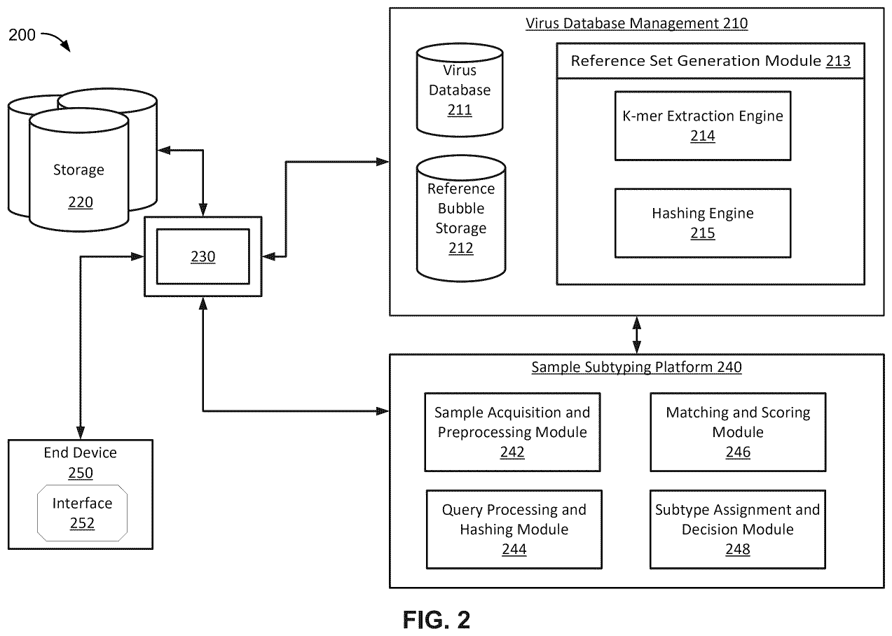

FIG. 2 shows an exemplary environment 200 for performing virus subtyping using k-mer hashing techniques. The environment 200 includes a virus database management platform 210, a storage 220, a network 230, a sample subtyping platform 240, and an end device 250. The virus database management platform 210 and the sample subtyping platform 240 can be implemented using software only (e.g., each module of the platform is a digital entity implemented using programs, code, or instructions executable by one or more processors), using hardware (e.g., a medical tool to perform probe testing, a sequencer, a GPU, a CPU, or the like), or using a combination of hardware and software. Although FIG. 2 illustrates a particular set and arrangement of the components, it should be understood that any suitable number or configuration of components may be included in the environment 200. Additional components such as various sequencing systems, cloud-based data repositories, or parallel computing resources may also be integrated as appropriate for specific implementations. Security measures, such as encrypted data transmission and user authentication, can be implemented to protect sensitive clinical and genomic information during processing and data sharing.

### Virus Database Management Platform 210

The virus database management platform 210 is a core component that provides the infrastructure and computational tools necessary for organizing, maintaining, and preparing viral sequence data for downstream subtyping analysis. As shown in FIG. 2, the virus database management platform 210 includes a virus database 211, a reference bubble storage 212, and a reference set generation module 213, which itself incorporates a k-mer extraction engine 214 and a hashing engine 215. Each of these subcomponents performs coordinated functions to support the efficient preparation, storage, and retrieval of reference datasets for use in the virus subtyping workflow.

The virus database 211 is configured for storing virus information, e.g., collections of complete viral genome sequences covering a variety of strains and subtypes, with each sequence annotated by its corresponding subtype or genotype label. Each genome sequence in the virus database 211 is annotated with a corresponding subtype or genotype label, and in some instances, only high-quality, complete sequences are retained to ensure the reliability and specificity of downstream subtyping processes. The virus database 211 can be deployed using local servers, network-attached storage devices, or cloud-based data warehousing solutions. Various database management systems may be used, including relational databases such as PostgreSQL and MySQL, or scalable NoSQL architectures, selected based on requirements for performance, scalability, and data accessibility. The virus database 211 is routinely updated to incorporate newly validated strains, recently discovered subtypes, and improvements from ongoing curation, thereby enabling the system to adapt to new developments and expansions in publicly available viral sequence repositories.

The reference bubble storage 212 is configured to serve as a repository for precomputed sets of hashed k-mers, referred to as reference bubbles. For each viral strain cataloged in the virus database 211, a corresponding reference bubble is created by extracting all possible k-mers of a specified length (e.g., k=15) from the complete genome sequence and hashing each k-mer to generate a digital signature unique to that strain. The resulting reference bubbles are stored in data structures designed for rapid access and efficient membership checks, such as hash tables or hash sets, supporting position-independent comparison between query samples and reference strains. The reference bubble storage 212 can be implemented using server-based memory (RAM), solid-state drives (SSD), high-throughput network-attached storage devices, or distributed object storage systems, depending on throughput and scalability requirements. Reference bubble storage 212 is updated in coordination with the virus database 211 to reflect the addition of new strains, subtypes, or improvements from curation, thereby ensuring the system remains current with advances in viral genomics and public sequence repositories.

The reference set generation module 213 is configured to coordinate the construction, maintenance, and periodic updating of reference bubbles for the virus subtyping workflows. The reference set generation module 213 integrates specialized computational engines and may be implemented using software applications, dedicated hardware, or a hybrid combination, depending on the deployment requirements and throughput demands. Within the reference set generation module 213, there is a k-mer extraction engine 214 configured to process each complete viral genome stored in the virus database 211. This a k-mer extraction engine 214 systematically extracts all possible k-mers of a specified length, such as 15-mers, from each genome sequence. The k-mer extraction engine 214 may be realized as a software module developed in Python, C++, or similar programming languages, or accelerated by hardware platforms including multicore CPUs, GPUs, or field-programmable gate arrays (FPGAs) to support high-throughput and large-scale processing. In some embodiments, commercial bioinformatics packages or custom-built algorithms may be deployed alongside laboratory sequencing instruments to automate extraction and preprocessing of viral sequence data.

The hashing engine 215 receives the output from the k-mer extraction engine 214 and applies a computational hash function to each extracted k-mer. This hash function converts each k-mer into a compact digital signature that enables rapid and efficient comparison during downstream analysis. The hashing engine 215 may use widely adopted cryptographic or non-cryptographic algorithms, and is compatible with other bioinformatic frameworks. The hashing engine 215 itself can be implemented in software, hardware accelerators, or a combination thereof, depending on performance needs. The resulting set of hashed k-mers for each genome is stored as a reference bubble within the reference bubble storage 212, using data structures such as hash tables or hash sets that are optimized for fast membership queries and efficient retrieval during subtyping operations.

In the context of environment 200, the term “engine” (for example, k-mer extraction engine 214 or hashing engine 215) refers to a dedicated processing unit, which may be realized as a specialized software module, a hardware accelerator, or a purpose-built circuit tasked with executing a specific computational function. The term “module” (such as reference set generation module 213) indicates a broader functional block that may encompass multiple engines or submodules, coordinating a sequence of operations to achieve the desired data preprocessing and encoding.

The reference set generation module 213 is designed to work in close integration with both the virus database 211 and the reference bubble storage 212. This tight coupling ensures that new viral strains, updated genome sequences, or additional subtypes are systematically processed and incorporated into the subtyping workflow. Automated update mechanisms may be employed to periodically refresh the reference bubbles in accordance with changes in public viral sequence repositories or laboratory findings. The reference set generation module 213 is also configured to interact with other components of environment 200, including the sample subtyping platform 240, storage 220, network 230, and end devices 250, facilitating seamless data exchange, distributed processing, and remote system access for a range of operational settings.

The virus database management platform 210 is configured to interact with other components of environment 200, including the sample subtyping platform 240, storage 220, network 230, and end device 250. Through these interactions, the virus database management platform 210 supplies reference datasets and annotation metadata for the subtyping analysis, receives updates and new sequence data, and supports distributed, cloud-based, or hybrid deployments. Data exchange between modules can occur over standard network protocols, high-speed data buses, or cloud application programming interfaces, depending on the system architecture. The virus database management platform 210 may be deployed as a combination of software applications, such as Python scripts, Docker or Conda environments, or as integrated systems that combine software with dedicated hardware resources including CPUs, GPUs, or high-performance storage appliances. This modular and scalable architecture enables the virus database management platform 210 to support subtyping for a range of viruses, accommodate changes in reference data as new strains and subtypes are discovered, and integrate with high-throughput sequencing instruments or automated update mechanisms.

### Storage 220

Storage 220 is configured to function as a central repository for large volumes of viral genomic sequence data, reference datasets, analytical outputs, and associated metadata within environment 200. Storage 220 supports the operational requirements of both the virus database management platform 210 and the sample subtyping platform 240, providing seamless integration and accessibility for all data necessary to support k-mer-based virus subtyping workflows.

In some embodiments, storage 220 may be implemented as a local storage system connected to network 230, enabling high-speed data transfer between the storage 220 and other components of environment 200. This architecture allows efficient access and retrieval of data, minimizing latency and supporting rapid query responses during subtyping analysis. Storage 220 can be realized using different technologies, such as direct-attached storage, network-attached storage (NAS), or cloud-based data warehousing solutions, selected according to data scale, throughput, and accessibility requirements.

The repository managed by storage 220 may include raw viral sequencing data, curated genome collections, precomputed reference bubble sets, and intermediate or final analysis results produced by the sample subtyping platform 240. By archiving both raw and processed datasets, storage 220 ensures data integrity, traceability, and availability throughout the entire virus subtyping pipeline. In some embodiments, storage 220 may also house computational models, bioinformatics tools, and algorithms required for data preprocessing, k-mer extraction, hashing, scoring, and subtype assignment. These resources may be accessed and executed directly within the storage infrastructure to support advanced analysis tasks, such as pattern recognition, recombination detection, and variant classification.

To facilitate accessibility and usability, storage 220 may be configured to support secure, real-time connections with the end device 250. This enables authorized users, such as laboratory technicians, clinicians, or researchers, to interact with stored data, retrieve curated datasets, and access analytical tools from remote locations. Security measures, including encrypted data transmission, user authentication, and role-based access control, are implemented to protect sensitive information and ensure that only authorized personnel can access or modify the stored data.

In some embodiments, storage 220 may be realized as a cloud-based storage solution integrated with network 230. Cloud-based deployment offers advantages such as scalability to accommodate expanding data volumes, redundancy for enhanced data protection, and global accessibility for collaborative research or distributed diagnostic workflows. When configured within a distributed architecture, storage 220 enables efficient data sharing, remote processing, and real-time collaboration among multiple users and devices. Whether deployed locally or in the cloud, storage 220 provides robust and secure management of viral genomic data, reference sets, and analytical outputs, supporting the performance and reliability of environment 200 across diverse operational scenarios.

### Network 230

Network 230 is configured to provide robust, high-speed, and secure data communications among the components of environment 200, including storage 220, the virus database management platform 210, the sample subtyping platform 240, and the end device 250. Network 230 supports a range of modem networking protocols and architectures, enabling reliable connectivity and efficient data transfer to facilitate the k-mer-based virus subtyping workflows illustrated in FIGS. 2-5.

Network 230 may be implemented as a local area network (LAN), a wide-area network (WAN), or a combination of public and private networks, including the Internet, virtual private networks (VPNs), or dedicated research networks. Contemporary network protocols such as TCP/IP, Ethernet, and advanced wireless standards (for example, Wi-Fi 6, Wi-Fi 7, or 5G cellular networks) are supported to provide high bandwidth, low latency, and secure transmission of large volumes of genomic data and analytical results. The network 230 can also integrate optical fiber links and high-throughput backbone connections where ultra-fast data movement is required, such as for distributed storage clusters or remote laboratory facilities.

To connect the virus database management platform 210, storage 220, the sample subtyping platform 240, and the end device 250 to network 230, various types of physical and wireless links may be employed. Wireline connections such as Ethernet, fiber optic cables, or DOCS cable modems can deliver reliable and scalable connectivity. Wireless solutions including Wi-Fi, 5G, and Bluetooth can enable mobility, remote access, and ease of installation. For geographically distributed deployments, network 230 may further integrate cloud-based networking services, software-defined networking (SDN), and edge computing nodes to optimize data routing and processing efficiency.

The integration of these diverse connection methods ensures a resilient and high-performance data communication framework. Network 230 enables seamless data exchange and real-time interaction among all components of environment 200, supporting complex workflows and large-scale analysis tasks required for virus subtyping. Security features such as encrypted data transmission, multi-factor authentication, and firewall protections are incorporated to safeguard sensitive patient and genomic data during transfer and remote access.

### Sample Subtyping Platform 240

The sample subtyping platform 240 is configured to process test samples and assigning subtypes using k-mer-based hashing techniques. As depicted in FIG. 2, the sample subtyping platform 240 comprises several specialized modules: the sample acquisition and preprocessing module 242, the query processing and hashing module 244, the matching and scoring module 246, and the subtype assignment and decision module 248. These modules may be realized in software, hardware, or a hybrid configuration, and are designed to support high-throughput, accurate, and automated subtyping workflows.

The sample acquisition and preprocessing module 242 is configured to receive and prepare viral nucleotide sequence data sourced from a broad spectrum of environments, including clinical patient samples, environmental collections, and research-derived specimens. The sample acquisition and preprocessing module 242 is engineered to accommodate diverse data formats, such as raw sequencing reads in FASTQ format produced by high-throughput sequencing instruments, as well as consensus sequences in FASTA format generated by upstream sequence assembly or computational preprocessing workflows.

To enable seamless integration with laboratory operations, the sample acquisition and preprocessing module 242 is designed to interact directly with a variety of sequencing modules or sequencers. This interaction may be realized through data transfer protocols and integration with the output systems of next-generation sequencing (NGS) instruments, Sanger sequencers, or other automated sequencing platforms. The sample acquisition and preprocessing module 242 can automatically retrieve sequencing files and associated metadata using direct USB, Ethernet, Wi-Fi, or through connections established with laboratory information management systems (LIMS) that aggregate data across multiple instruments. In some embodiments, sequencing platforms may be physically linked to dedicated processing servers or high-performance workstations where sample acquisition and preprocessing module 242 is installed, while in other scenarios, data may be routed through secure cloud-based storage or network-attached servers for centralized access and processing.

Upon receiving input data from sequencing modules or sequencers, the sample acquisition and preprocessing module 242 performs a comprehensive suite of preprocessing steps to ensure that the sequence data meets the quality and formatting requirements for downstream k-mer analysis. These preprocessing routines may include quality filtering to remove reads or bases below a specified confidence threshold, adapter and barcode trimming to eliminate sequencing artifacts, and error correction algorithms to address ambiguities or errors introduced by the sequencing process. The sample acquisition and preprocessing module 242 is further capable of executing format conversions, ensuring compatibility with downstream modules in the sample subtyping platform 240, regardless of the original output format of the sequencing platform.

The functions of the sample acquisition and preprocessing module 242 may be performed by a range of physical components, including dedicated data processing servers, high-performance laboratory workstations, or cloud-based virtual machines equipped with robust storage and computational resources. Solid-state drives (SSDs), high-throughput network interfaces, and multicore CPUs or GPUs may be employed to facilitate rapid data ingestion, preprocessing, and transfer. For laboratories with significant data volumes or real-time processing needs, the sample acquisition and preprocessing module 242 may also be realized as a hardware-accelerated appliance or integrated into automated laboratory robotics systems.

The sample acquisition and preprocessing module 242 is also designed to connect with automated sample tracking platforms, sequencing instrument control systems, or LIMS. This integration allows for automated intake of sample identifiers, association of relevant metadata, and real-time tracking of sample status throughout the analysis pipeline. Such features streamline the sample intake process, reduce the likelihood of manual data entry errors, and improve traceability and provenance of each sample for downstream quality assurance.

To further enhance the reliability and reproducibility of the virus subtyping workflow, the sample acquisition and preprocessing module 242 may implement automated quality control (QC) procedures. These procedures can include assessments of sequence coverage, evaluation of base quality distributions, and detection of potential contaminants, chimeric reads, or unexpected sequence artifacts. QC metrics and processing results generated by the sample acquisition and preprocessing module 242 can be logged, reported, and used to flag problematic samples, enabling laboratory personnel or automated monitoring systems to intervene before data is advanced to subsequent modules within the sample subtyping platform 240.

The query processing and hashing module 244 is configured to process input viral nucleotide sequence data by extracting k-mers of a specified length and generating a corresponding test set of hashed k-mers for each sample. This step establishes the digital fingerprint for each query sequence, enabling rapid and robust comparison to reference datasets.

Upon receiving preprocessed sequence data from the sample acquisition and preprocessing module 242, the query processing and hashing module 244 systematically enumerates all possible k-mers from each input query sequence. The extraction process is designed to be robust to sequence variability and can handle both raw sequencing reads and consensus sequences. To support high-throughput and large-scale operations, the query processing and hashing module 244 may be implemented as optimized software pipelines written in programming languages such as Python or C++, or may be executed on high-performance computing platforms including multicore CPUs, GPUs, or FPGAs. For laboratories with extensive data processing needs, dedicated hardware appliances or parallelized cloud computing nodes may be employed to accelerate extraction and hashing tasks.

Following k-mer extraction, the query processing and hashing module 244 applies a computational hash function to each k-mer to generate a compact digital representation, or hash value, for every k-mer in the query sequence. The same hash function used in the reference set generation module 213 is applied, ensuring consistency in the digital encoding and compatibility with the reference bubbles stored in the reference bubble storage 212. The hash function may be selected from widely adopted cryptographic or non-cryptographic algorithms, and is compatible with different bioinformatic frameworks. The resulting set of hashed k-mers forms the test set that will be used for subsequent matching and scoring operations.

The query processing and hashing module 244 is further designed for flexibility and integration with other components of the sample subtyping platform 240. Data structures such as hash tables or hash sets may be used to organize and store the hashed k-mers, supporting rapid membership queries during the downstream comparison step. In advanced configurations, the module 244 may employ commercial bioinformatics software packages, custom-built analysis tools, or distributed processing frameworks that interface seamlessly with laboratory information management systems, sequencing data management platforms, and cloud-based storage solutions. In addition to its primary extraction and hashing functions, the query processing and hashing module 244 can be programmed to generate metadata or summary statistics, such as the total number of unique k-mers extracted, coverage depth, or frequency distributions of observed k-mers. These metrics can be logged, reported, or used for downstream quality control and troubleshooting.

The modular design of the query processing and hashing module 244 enables it to be deployed in a range of operational environments, including centralized clinical laboratories, distributed research networks, and automated point-of-care diagnostic systems. Whether realized as a software pipeline on a general-purpose server, as a GPU-accelerated application for high-throughput settings, or as a cloud-based service, the query processing and hashing module 244 ensures that each viral sample is consistently and efficiently encoded for accurate and scalable virus subtyping

The matching and scoring module 246 is configured to perform comparing each test set of hashed k-mers generated from a sample to the precomputed reference sets stored in the reference bubble storage 212. The matching and scoring module 246 operates in a position-independent manner, meaning that matching is determined solely by the presence of identical hashed k-mers in both the test set and a given reference set, regardless of the original genomic locations of those k-mers. This approach allows for highly efficient and scalable comparison across large reference databases and supports robust subtyping even in the presence of sequence rearrangements or genome variability.

Upon receiving the test set of hashed k-mers from the query processing and hashing module 244, the matching and scoring module 246 systematically queries each reference bubble stored in the reference bubble storage 212. For each reference set, the module 246 determines the overlap between the hashed k-mers of the sample and those of the reference, identifying all matches and mismatches. To optimize performance and throughput, this process can be implemented using efficient data structures such as hash tables or hash sets, and may be accelerated using high-performance computing resources, including multicore CPUs, GPUs, or parallelized cloud-based processing nodes. For laboratories with extensive throughput requirements, dedicated hardware appliances or FPGAs may further enhance the speed and efficiency of these operations.

The matching and scoring module 246 is configured to implement a scoring algorithm. For each reference set, the matching and scoring module 246 computes a k-mer matching score by assigning a positive weight to each matching k-mer (e.g., equal to the length of the k-mer) and applying a penalty (e.g., −1) to each unmatched k-mer. This weighted approach quantitatively reflects the degree of similarity between the sample's sequence data and each reference genome, supporting sensitive discrimination among closely related viral subtypes. The scoring algorithm is robust to sequence variability and is designed for rapid computation across large datasets. In some embodiments, different scoring algorithms are stored in the matching and scoring module 246.

In addition to the raw k-mer matching scores, the matching and scoring module 246 may calculate further metrics, such as the total number of matching k-mers, the proportion of unique k-mers shared, and the cumulative score across all reference sets. These metrics are used in downstream steps to derive the query identity, which is the percentage of bases in the reference genome covered by matching k-mers from the sample. In some embodiments, the query identity is calculated in this matching and scoring module 246 as a metric. All computed scores and metrics are passed to the subtype assignment and decision module 248 for subsequent processing.

To support transparency and reproducibility, the matching and scoring module 246 can be programmed to log detailed results of each comparison, including lists of matching and non-matching k-mers, intermediate scores, and quality control metrics. In advanced implementations, this module may also interface with laboratory information management systems, cloud-based analytics dashboards, or automated reporting pipelines to facilitate results interpretation and quality assurance.

The subtype assignment and decision module 248 is configured to analyze the results of the k-mer matching and scoring operations and for determining the most probable viral subtype for each processed sample. The subtype assignment and decision module 248 applies a structured decision-making framework that incorporates both quantitative and rule-based approaches to maximize the reliability and interpretability of the subtyping outcome.

Upon receiving matching scores and related metrics from the matching and scoring module 246, the subtype assignment and decision module 248 calculates the query identity for each candidate reference strain. The query identity is defined as the percentage of bases in the reference genome that are covered by matching k-mers from the test set. This metric provides a direct measure of similarity between the sample and each reference strain, supporting nuanced discrimination among closely related viral subtypes.

The subtype assignment and decision module 248 then applies a filtering step, retaining only those reference strains with a query identity above a predefined threshold, such as 80 percent. This threshold ensures that only highly similar reference strains are considered in the final subtype determination, reducing the risk of erroneous or ambiguous calls. Up to a set number of top candidate strains, for example, four, may be selected for final consideration based on their query identity values.

The decision-making process in the subtype assignment and decision module 248 follows a two-tiered approach. If all top candidate strains share the same subtype or genotype label, the subtype assignment and decision module 248 assigns that subtype to the sample with high confidence, reflecting a consensus among the highest-scoring matches. This assignment is reported along with a categorical confidence level (e.g., “higher” confidence), supporting clinical or research decisions that depend on highly reliable subtyping information.

If the top candidate strains do not all agree on a single subtype, the subtype assignment and decision module 248 employs a weighted voting algorithm. Each candidate subtype is assigned a weight (e.g., equal to the sum of the query identity scores of strains in that subtype category). The subtype assignment and decision module 248 then selects the subtype with the highest aggregated score as the primary assigned subtype with a confidence level (e.g., “high” or “medium” or “1” or “2”), while also reporting the secondary closest subtype (the one with the second highest total weight) and corresponding confidence levels (e.g., “low” or “lower”). This approach provides transparency and supports nuanced interpretation in cases where the sample exhibits sequence similarity to multiple subtypes, such as in recombinants or mixed infections.

The subtype assignment and decision module 248 may further log and report all decision-relevant metrics, including the list of candidate strains, their query identities, assigned weights, confidence levels, and any secondary subtype assignments. Additionally, the subtype assignment and decision module 248 is designed to be integrated with laboratory information management systems, analytical dashboards, or automated reporting tools, enabling seamless export of results and metadata to end users and supporting systems. Security and privacy measures, such as user authentication and access control, may be employed to ensure that sensitive sample results are protected during reporting and transmission.

### End Device 250

The end device 250 is an electronic device comprising hardware, software, embedded logic components, or a combination of these elements, and is configured to interact with the virus database management platform 210, storage 220, the sample subtyping platform 240, and network 230. The end device 250 may include a range of contemporary computing systems such as desktop computers, laptops, workstation computers, tablets, smartphones, portable handheld devices, wearable computing devices, thin clients, or other specialized laboratory or clinical terminals. These computing devices can run various operating systems and application environments, including Windows, macOS, Linux distributions, Android, iOS, or other modern or embedded operating systems.

The end device 250 may be designed to execute a variety of client-side or web-based applications that support user interaction with the virus subtyping pipeline. For example, the end device 250 may run specialized software for submitting viral sequence data, reviewing subtyping analysis reports, managing database updates, or accessing curated reference datasets. The end device 250 may also support secure user authentication, audit trails, and role-based access control, ensuring that only authorized users can access sensitive genomic and diagnostic information.

The end device 250 comprises an interface 252, such as a graphical user interface (GUI), which enables users to interact intuitively with the environment 200. Through interface 252, users can upload sequence data, initiate new subtyping analyses, visualize subtyping results, monitor workflow status, or configure system parameters. The interface 252 may support advanced visualization tools for exploring k-mer matching profiles, confidence scores, or recombination analysis, and may also integrate with LIMS or electronic medical records for seamless data exchange.

The end device 250 is capable of both inputting and receiving data over the network 230. For example, a laboratory technician, clinician, or researcher may use the end device 250 to submit viral nucleotide sequence data or analysis requests to the sample subtyping platform 240. The end device 250 may also be used to retrieve subtyping results, download analytical reports, or access the latest updates to reference databases maintained by the virus database management platform 210. Data transmission between the end device 250 and other components may occur via wired connections (such as Ethernet or USB) or via wireless protocols (such as Wi-Fi, Bluetooth, or cellular networks), depending on the deployment scenario.

In some embodiments, the end device 250 may also support integration with cloud-based services or distributed computing environments. This enables remote access to the virus subtyping system, supports telemedicine or distributed research collaborations, and allows authorized users to interact with the environment 200 from virtually any location. Security features, including encrypted data transfer, multi-factor authentication, and digital certificates, may be implemented to protect sensitive patient, sample, and genomic data during transmission and remote access.

The end device 250 may further incorporate notification systems, audit logs, and automated reporting tools, enabling users to receive alerts about workflow completion, system updates, or quality control events. Advanced deployments may allow the end device 250 to interface with laboratory automation platforms, robotic sample handlers, or sequencing instruments for fully automated, end-to-end virus subtyping workflows.

In some embodiments, environment 200 may be further augmented with additional components designed to enhance performance, scalability, and adaptability for advanced virus subtyping workflows. For example, high-throughput sequencing systems can be incorporated to generate large volumes of raw viral sequence data from clinical, environmental, or research samples. These sequencing systems may be directly connected to the sample acquisition and preprocessing module 242, enabling automated transfer of sequencing outputs into the sample subtyping platform 240 for immediate downstream processing. Sequencing instruments may be physically located in laboratory environments and interfaced with the network 230 for seamless data integration and real-time analysis.

Parallel computation resources, such as GPU clusters, multicore CPU servers, or cloud-based high-performance computing environments, may also be deployed within environment 200 to accelerate computationally intensive steps. These resources can be allocated to the query processing and hashing module 244 and the matching and scoring module 246 within the sample subtyping platform 240, enabling rapid extraction, hashing, and comparison of k-mers even when processing large sample batches or extensive reference databases. Parallel computing capabilities may also support the reference set generation module 213 within the virus database management platform 210, expediting the construction and updating of reference bubbles as new viral strains are incorporated.

In some embodiments, the computational steps for k-mer extraction, hashing, and comparison may be parallelized across multiple processing nodes within a high-performance computing cluster or distributed computing environment. For example, large batches of query samples may be divided among several servers, with each server independently performing k-mer extraction and hash generation for its assigned subset of samples. The resulting hashed k-mer sets may then be distributed for simultaneous comparison to different partitions of the reference database, enabling concurrent matching and scoring operations. This distributed architecture supports horizontal scaling, so that as the number of samples or reference strains increases, additional processing nodes can be added to maintain high throughput and rapid turnaround times. Such parallelization is particularly advantageous in clinical laboratories or surveillance settings that require the processing of thousands of samples per day, or in research environments analyzing large genomic datasets across multiple viral species.

Automated update mechanisms can be integrated to refresh the virus database 211 and reference bubble storage 212 as new viral genome sequences, strains, or subtypes become publicly validated or internally curated. These mechanisms may interact with external sequence repositories, laboratory data management systems, or bioinformatics pipelines, orchestrating scheduled or event-driven updates to maintain the accuracy and comprehensiveness of the reference datasets. Updates may be triggered on a periodic basis (such as quarterly or annually) or in response to specific events, such as the publication of new viral genome data or the identification of emergent viral subtypes. The update processes are coordinated through the reference set generation module 213 and may leverage the computational and storage infrastructure of storage 220 and network 230 to ensure efficient data synchronization and minimal disruption to ongoing analyses.

The modular architecture of environment 200 enables flexible scaling and adaptation to a variety of laboratory, clinical, or research settings. Components such as storage 220 and network 230 are designed to support distributed processing, remote access, and collaborative workflows, allowing laboratories to accommodate increasing data volumes, support geographically dispersed teams, or integrate with external diagnostic networks. The end device 250, equipped with the interface 252, provide access points for laboratory personnel, clinicians, or researchers to monitor system operations, initiate analyses, review results, and manage database updates from local or remote locations.

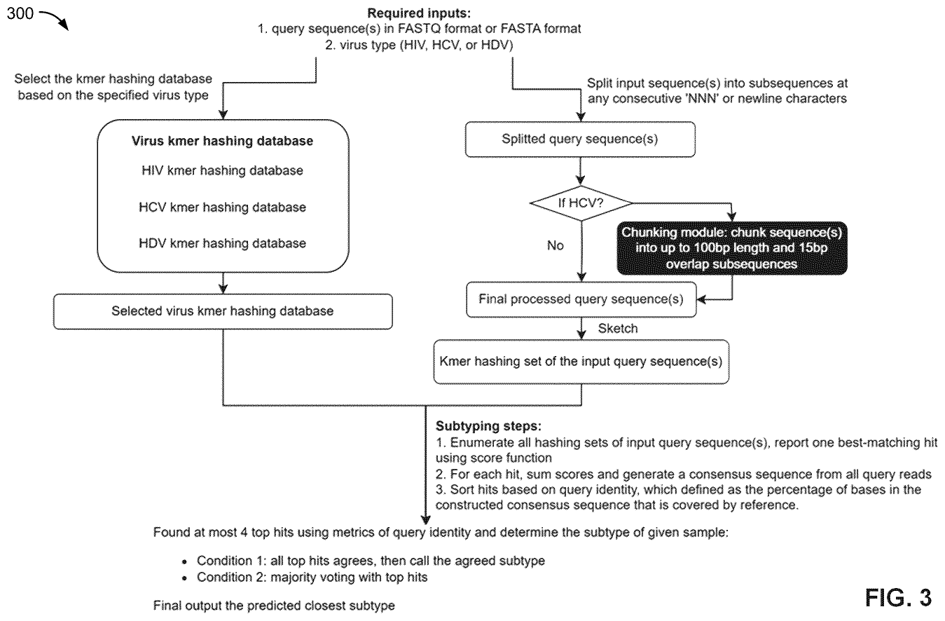

In some embodiments, virus subtyping workflows (see e.g., workflows in FIGS. 3-5) may be deployed in a cloud computing environment, such as a public or private cloud platform. In such implementations, components such as the virus database management platform 210, reference bubble storage 212, and sample subtyping platform 240 may be instantiated as scalable microservices or virtual machines. Cloud orchestration tools, such as Kubernetes or Docker Swarm, can dynamically allocate computational resources based on demand, automatically scaling the number of active processing instances in response to sample volume or database growth. The virus database 211 and the reference bubble storage 212 may be stored in distributed object storage, while matching and scoring tasks are assigned to serverless compute functions or containerized workloads operating in parallel. This architecture supports geographically distributed laboratories, enabling users to submit sequencing data from remote locations and receive subtyping results with minimal latency. Additionally, cloud-based deployment facilitates real-time collaboration, data sharing, and integration with external analytical pipelines, while ensuring data security and compliance through encryption, authentication, and role-based access controls.

In some embodiments, a hybrid deployment model may be adopted, in which pre-processing and initial k-mer extraction are performed on local laboratory servers, while computationally intensive matching and scoring steps are offloaded to cloud-based resources. This hybrid approach allows laboratories to retain control over sensitive patient data during acquisition and pre-processing, while taking advantage of the virtually unlimited compute and storage capacity of the cloud for large-scale k-mer comparison and reference database management. Results and supporting metrics are returned to the local environment for review, reporting, and integration with LIMS.

### IV. EXAMPLE WORKFLOW 1

FIG. 3 illustrates an exemplary workflow 300 for a k-mer-based virus subtyping technique, showing the primary data processing path and decision logic used to classify viral samples by subtype. This workflow 300 can be implemented using the integrated system components described in FIG. 2, with each step corresponding to specific modules and operations in environment 200.

### Input Requirements and Initial Selection

At the top of FIG. 3, the required inputs for the workflow 300 include: (1) one or more query sequences provided in FASTQ format (raw sequencing reads) or FASTA format (consensus sequences), and (2) the virus type to be analyzed, such as HIV, HCV, or HDV. These inputs may be received and preprocessed by the sample acquisition and preprocessing module 242 of the sample subtyping platform 240.

The workflow 300 proceeds by selecting the appropriate virus k-mer hashing database based on the specified virus type. The virus k-mer hashing databases (for example, HIV, HCV, or HDV) are pre-built reference databases, each comprising comprehensive collections of hashed k-mers for all reference strains. These databases are stored and maintained within the virus database 211 and reference bubble storage 212, as depicted in the virus database management platform 210 of FIG. 2.

### Preprocessing and Query Sequence Handling

Input query sequences can be split into subsequences at any consecutive “NNN” nucleotide stretches or newline characters, which helps address sequence gaps or ambiguous regions commonly found in sequencing data. This splitting can be performed within the sample acquisition and preprocessing module 242, ensuring that each segment is suitable for downstream hashing and comparison.

For HCV samples, the workflow 300 incorporates a chunking module, which further divides each query sequence into chunks of up to 100 base pairs, with a 15 base pair overlap between consecutive chunks. This process increases sensitivity for detecting HCV recombinants and improves subtyping resolution for highly variable or recombinant viral genomes. The chunking logic is executed within or in conjunction with the sample acquisition and preprocessing module 242.

### K-mer Hashing and Test Set Construction

After preprocessing, the resulting query sequence(s), whether split, chunked, or directly passed through, are processed to generate a k-mer hashing set. This may be performed by the query processing and hashing module 244, which systematically extracts all k-mers of the predefined length (15-mers) from each processed query segment and applies a hash function to create compact digital representations for each k-mer. The resulting k-mer hashing set forms a digital fingerprint of the sample, which is then compared against the selected virus k-mer hashing database.

### Matching, Scoring, and Subtype Assignment

The k-mer hashing set of the input query sequence(s) is compared to the reference k-mer hashing sets in the selected virus k-mer hashing database. For each reference strain, the workflow 300 computes a matching score by enumerating all matches and mismatches between the hashed k-mers of the query and the reference. This matching and scoring can be performed by the matching and scoring module 246 within the sample subtyping platform 240.

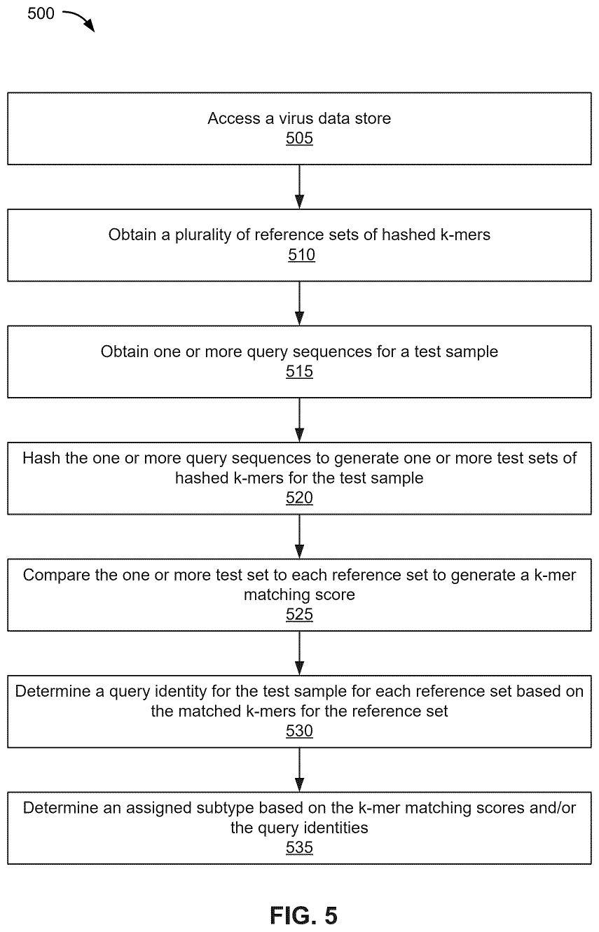

Each reference strain is scored, and a consensus sequence may be generated from all query reads by aggregating the results of k-mer matches. The workflow 300 then sorts the reference strains based on query identity, which is defined as the percentage of bases in the constructed consensus sequence that are covered by reference k-mers. This calculation is consistent with the process 500 described for query identity determination in FIG. 5, e.g., at block 530.

### Subtyping Logic and Output

The workflow 300 identifies up to four top candidate reference strains with the highest query identity metrics. The subtype assignment and decision module 248 in FIG. 2 may be used to determine the subtype of the sample using a set of decision rules including:
Condition 1: If all top hits agree on the same subtype, the agreed subtype is assigned to the sample with high confidence;
Condition 2: If the top hits do not all agree, a majority voting or weighted voting process is applied among the top hits.

The subtype with the largest aggregated score (e.g., the sum of query identities) is assigned as the predicted closest subtype. The workflow 300 may also report the second closest subtype and relevant confidence levels, supporting nuanced and transparent reporting. The final output is the predicted closest subtype for the sample, along with supporting metrics such as query identity, matching scores, and confidence levels. This output can be reviewed and reported to users through the end device 250 and its interface 252, as shown in FIG. 2.

Integration with System Architecture

The workflow 300 shown in FIG. 3 may operate within the framework of environment 200 in FIG. 2, relying on the virus database management platform 210 for reference data, the storage 220 for managing large-scale sequence and result data, the network 230 for communication, and the sample subtyping platform 240 for executing the computational and decision-making steps. This modular and extensible architecture supports high-throughput, accurate, and scalable virus subtyping for clinical diagnostics, surveillance, and research applications.

It should be understood that the numbers, thresholds, and metrics illustrated in FIG. 3, such as the specific chunk size, overlap, k-mer length, threshold for query identity, and the number of top candidate strains considered for subtype assignment, are provided as exemplary values. These values are selected to illustrate the workflow 300 and decision logic, but should not be construed as limiting the scope of the disclosed techniques. Alternative values, thresholds, and operational parameters may be used depending on the virus type, sequencing technology, or specific deployment scenario, without departing from the scope of the disclosed subject matter.

### V. EXAMPLE WORKFLOW 2

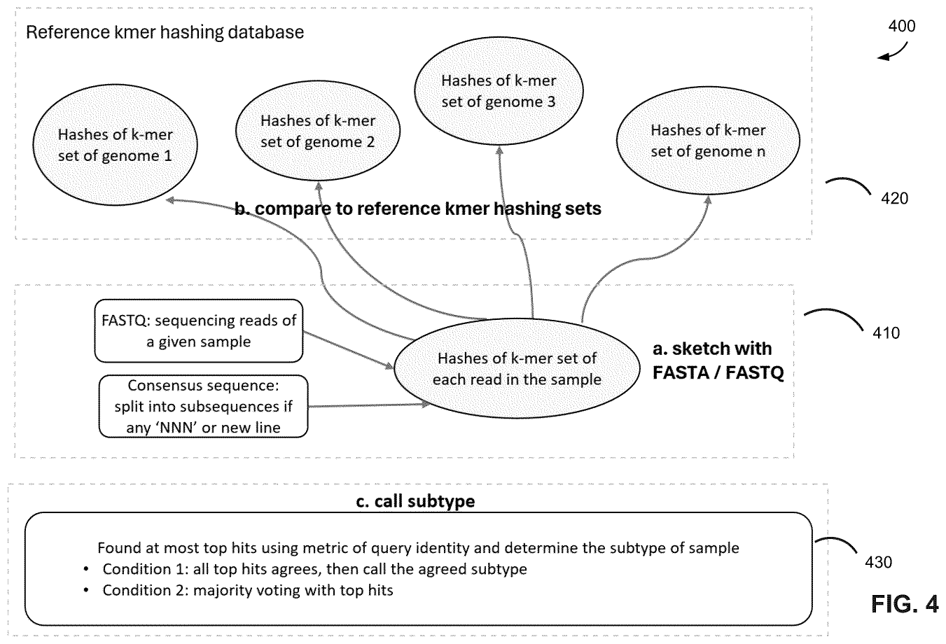

FIG. 4 illustrates a high-level schematic workflow 400 for virus subtyping using a k-mer hashing approach. The workflow 400 provides an overview of how input sample data is processed and compared to a reference k-mer hashing database to assign a viral subtype. The steps shown in FIG. 4 correspond to the primary functions performed by components such as the sample subtyping platform 240 and the virus database management platform 210 in environment 200 described in FIG. 2, and align with the process flow blocks shown in FIG. 5.

Step 410: Sketch with FASTA/FASTQ

At step 410, the workflow 400 begins with the input of sample data, which can be provided as raw sequencing reads in FASTQ format or as consensus sequences in FASTA format. If consensus sequences are used, they are split into subsequences at any occurrence of “NNN” or newline characters, to address ambiguous or missing bases in the sequence. The sample acquisition and preprocessing module 242 within the sample subtyping platform 240 can be used for handling these input formats and preprocessing steps.

Once preprocessing is complete, k-mer sketching is performed. For both FASTQ and FASTA inputs, the k-mer hashing set is generated by extracting all k-mers of a specified length (e.g., 15-mers) from the processed sequence data and hashing each k-mer to produce a digital signature. This operation can be carried out by the query processing and hashing module 244. The result is a set of hashed k-mers that represents the digital fingerprint of the input sample.

Step 420: Compare to Reference k-mer Hashing Sets

At step 420, the next stage of workflow 400 involves comparing the hashed k-mer set of the input sample to the reference k-mer hashing sets stored in the reference k-mer hashing database. This database comprises precomputed hashes of k-mer sets for each genome in the reference panel, as managed by the virus database 211 and reference bubble storage 212 within the virus database management platform 210.

The matching and scoring module 246 within the sample subtyping platform 240 may be used to perform this comparison, matching the sample's hashed k-mer set against each hashed k-mer set in the reference database. This matching is position-independent: a match is determined solely by the presence of an identical hashed k-mer in both the sample and a reference set, regardless of its original genomic location. For every reference genome, a similarity score or matching metric is calculated, e.g., by counting the number of matching hashed k-mers and applying a scoring algorithm that assigns a positive weight to matches and a penalty to mismatches. This comparison and scoring is performed for each genome in the reference database (e.g., genome 1, 2, 3, . . . n, as shown in FIG. 4), whether the database contains at least thousands of genomes corresponding to hundreds of HCV subtypes.

The process may further include the generation of a consensus sequence for the test sample, derived from the aligned k-mer matches, and the calculation of query identity for each genome. Query identity is typically defined as the percentage of bases in the reference genome that are covered by matching k-mers from the test set, supporting robust discrimination among closely related viral subtypes and genotypes.

### Step 430: Call Subtype

After matching at step 420, the workflow 400 proceeds to the subtype assignment step. The subtype assignment and decision module 248 may be used to filter the reference hits to retain the top candidates using a metric such as query identity, which is defined as the percentage of bases in the constructed consensus sequence that are covered by matching k-mers from the reference. The decision logic follows a two-condition approach: under Condition 1, if all top hits agree on the same subtype, that subtype is assigned to the sample with high confidence; under Condition 2, if the top hits do not all agree, a majority or weighted voting process is applied among the top hits, and the subtype with the highest aggregated score (typically the sum of query identities) is assigned as the predicted closest subtype.

Step 430 results in the final subtype call for the sample, along with supporting confidence metrics. The output, which includes the assigned subtype and supporting statistics, can be reported to users via the end device 250 and its interface 252, as shown in FIG. 2. This output may also be recorded in laboratory information management systems or analytical dashboards, supporting clinical, surveillance, or research applications.

### System Integration and Architecture

The workflow depicted in FIG. 4 operates within the broader architecture of environment 200, leveraging the storage 220 for management of sequence and analysis data, and the network 230 for data transfer between modules and user endpoints. The reference k-mer hashing database is maintained and updated by the virus database management platform 210, while the computational steps for sketching, matching, scoring, and subtype assignment are performed by the sample subtyping platform 240.

It should be understood that the metrics, such as the number of top hits, length of k-mers, and thresholds for subtype assignment, shown in FIG. 4 are provided as illustrative examples. The precise values, parameters, and decision logic may be adapted based on the specific virus, sequencing technology, or deployment scenario. Therefore, the depiction in FIG. 4 should not be interpreted as limiting the scope of the described methods or system. Instead, it is intended to illustrate the general workflow and integration of system components for k-mer-based virus subtyping.

### VI. EXEMPLARY PROCESS FOR VIRUS SUBTYPING USING K-MER HASHING TECHNIQUES

FIG. 5 is a flowchart illustrating a process 500 for performing virus subtyping using a position-independent k-mer hashing approach. The process 500 depicted in FIG. 5 may be implemented in software, such as code, instructions, or a program executed by one or more processing units, including processors or cores of a computing system, hardware, or combinations thereof. For example, the process 500 may be performed as part of an automated virus subtyping pipeline or computational platform designed to classify viral sequences based on k-mer content. The process 500 is intended to be illustrative and not limiting. Although FIG. 5 depicts the process steps in a particular order, the steps may be performed in different sequences, or some steps may be performed in parallel or substituted with alternative embodiments in certain implementations.

At block 505, a virus data store is accessed. The virus data store (e.g., a data repository or database) a plurality of virus strains representing a plurality of subtypes of the virus of interest (e.g., HIV). The virus data store may be accessed from a public genomic resource (e.g., a publicly available repository or database), a private repository or database, or an in-house database assembled for a specific application, depending on the requirements of the particular implementation. The virus data store is structured to include a comprehensive collection of complete or partial genome sequences for a wide variety of viral strains, with each entry annotated by a clearly defined subtype or genotype. For example, the virus data store may contain thousands of complete HIV genomes spanning over thirty recognized subtypes, thousands of HCV genomes across more than one hundred subtypes, as well as extensive collections for other clinically significant viruses such as HDV. In some embodiments, the virus data store is accessed to obtain a plurality of virus strains of the plurality of subtypes of the virus, with each virus strain of the plurality of virus strains having a genome sequence and an associated subtype annotation.

Virus data stored in the virus data store may be curated to ensure that each included strain sequence is both complete and reliably labeled with its respective subtype or genotype. For example, when a virus data store is accessed from an external source, such as a public genomic repository, a series of curation steps may be performed to validate the quality and accuracy of each entry. The curation process may begin by verifying that each sequence represents a complete genome or a sufficiently comprehensive partial genome required for subtype determination. Incomplete sequences, truncated records, or sequences with extensive ambiguous bases may be excluded to maintain the integrity of the reference set.

The curation process may also involve a thorough review of subtype or genotype annotations. This may include cross-referencing each label with standardized taxonomic resources or authoritative databases, resolving discrepancies in nomenclature, and updating subtype names to reflect current classification schemes. Strains lacking clear or consistent subtype labels may be flagged for manual review or removed from the accessed virus data store. Additionally, duplicate entries are identified and eliminated to prevent redundancy that could bias the matching process. Ambiguously labeled sequences, such as those with conflicting or uncertain subtype assignments, may be either corrected based on supporting information or excluded from the curated set. Harmonization of metadata, such as collection source, date, and geographic origin, may also be performed to facilitate downstream analysis and ensure traceability.

The virus data store can be designed for extensibility and periodic updating, so that as new viral strains, subtypes, or genotypes are validated and become available, the data store can be updated to ensure that process 500 remains current with scientific developments and evolving viral diversity. Additionally, the virus data store may be expanded to include viral genomes from other species, such as influenza viruses, coronaviruses, papillomaviruses, or flaviviruses, provided that comprehensive and curated sequence data are accessible. In some embodiments, a separate data store is constructed and stored for each virus. In some embodiments, a virus data store is constructed and stored for all viruses of interest.

Structurally, the virus data store is organized to support rapid and scalable computational comparison. Each viral strain's genome in the virus data store may be pre-processed, for example, in subsequent steps (see block 510) to extract all possible k-mers of a specified length, such as 15 nucleotides, and each k-mer is hashed using a selected hash function. The resulting set of hashed k-mers forms a digital fingerprint (e.g., a digital key or an index), also referred to as a reference set or reference bubble, for each strain. These reference sets are stored in digital data structures such as hash tables or sets, allowing for constant-time membership checks and efficient, position-independent comparison with query sequences during the subtyping workflow. The virus data store may be implemented in various formats, such as flat files, relational or NoSQL databases, or in-memory storage, to meet the computational infrastructure and scalability needs of the subtyping pipeline.

At block 510, a plurality of reference sets of hashed k-mers are obtained. Each reference set of the plurality of reference sets corresponds to a virus strain of a plurality of subtypes of a virus. The reference sets may be generated by performing a hash function on k-mers of the virus strains, and the generated hashed k-mer is stored as a digital key or index to store the corresponding k-mers. Virus data curation may also be performed at block 510. In some embodiments, only strains that meet specific qualification criteria, such as completeness of the genome and the presence of a reliable subtype or genotype label, are selected for processing. For each virus strain that meets the specific qualification criteria, a set of k-mers is generated by extracting nucleotide sequences of a specific length from the genome sequence using a sliding window. For example, if k is 15, the first k-mer consists of nucleotides 1 through 15, the second k-mer consists of nucleotides 2 through 16, the third k-mer consists of nucleotides 3 through 17, and so on, moving one nucleotide at a time along the sequence. This process continues until every possible contiguous k-mer has been extracted, ensuring comprehensive coverage of both conserved and variable sequence fragments that may contain subtype-informative signals.

An appropriate k-mer length is chosen to achieve the desired specificity and sensitivity for the viral species and/or subtyping resolution. In some embodiments, a preferred value of k is set to 15 across different viruses. Shorter k-mers (e.g., k<15) tend to produce non-unique matches, which can lead to overlaps across different subtypes. Longer k-mers (e.g., k>15) increase specificity but reduce sensitivity, especially for highly variable viral regions. 15-mers are long enough to uniquely characterize subtype-informative regions, while still tolerant of sequence variations.

In some embodiments, k is selected based on the virus, region, or application. In some embodiments, a shorter k value (e.g., k<15) may be selected to improve sensitivity when analyzing highly fragmented or degraded samples, or when the goal is to capture shared motifs across diverse viral populations. Conversely, a longer k value (e.g., k>15) may be chosen to increase specificity and reduce spurious matches when distinguishing closely related strains or when working with large, redundant reference databases. The choice of k can also be informed by the sequencing technology, read length, error profile, or the type of genetic event being detected, such as single nucleotide variants (SNVs), copy number variations (CNVs), or structural rearrangements. For certain diagnostic or prognostic applications, optimizing k may enhance detection accuracy or resolution at the desired taxonomic level. In some embodiments, k is set to an integer between 5 and 35, allowing the workflow to be adapted for a variety of viruses, genomic regions, and analytical objectives.

Extracted k-mers are then transformed into a digital representation by applying a selected hash function. The hash function may be a cryptographic hash function (e.g., polynomial rolling hash) such as SHA-256 or MD-5, or a custom non-cryptographic hash function optimized for computational efficiency and minimal collision rates. For example, if the extracted k-mer is “AGCTTAGCTTAGCTT,” instead of storing the sequence of characters in an array, the SHA-256 hash function is applied to this k-mer to produce a unique hexadecimal string representing the k-mer in digital form. Alternatively, if a polynomial rolling hash is used, each nucleotide can be assigned an integer value (such as A=0, C=1, G=2, T=3), and the hash value is computed by combining these values using a mathematical formula to generate a unique integer for the k-mer. The choice of hash function is made to ensure that each k-mer is converted into a unique or nearly unique value that facilitates rapid comparison and storage. In some embodiments, the choice of hash function is application-dependent and may be selected for computational efficiency, collision avoidance, or compatibility with existing workflows. In some embodiments, unique k-mers are selected and transformed into the digital representation.

In some embodiments, if the extracted k-mers are text strings, they may be converted into a stringed value (e.g., converting “ATCG” into “00011011” or “65846771”) or a value (e.g., an integer) may be assigned to each k-mer. The hash function is then applied to the assigned value to generate a hash digest (e.g., the digital key or index) and the hash digest is stored in the hash table or hash set representing the corresponding k-mer.

The resulting hashed k-mers are compiled for each strain and stored as a strain-specific reference set (i.e., the reference bubble), which serves as a digital fingerprint unique to that strain. These reference sets are further organized as hash tables or hash sets, which are digital data structures specifically designed for rapid membership queries. The use of a hash table enables each membership check (e.g., determining whether a particular hashed k-mer from a test set is present in a reference set) to be performed in constant time, or O(1), regardless of the size of the reference set or the overall virus database.

For example, consider a scenario where a test sample has thousands of hashed k-mers to be checked against a reference set containing millions of entries. If the reference set were stored as a simple list or array, each membership check would require scanning through the list, resulting in O(n) time complexity per query, where n is the number of entries (e.g., millions). In contrast, a hash table uses the hash value of each k-mer to instantly locate its presence or absence, allowing the same check to be completed in a fixed amount of time, independent of the set's size.

This efficiency is particularly beneficial in large-scale or production environments, where thousands of query k-mers must be compared across many reference sets (e.g., an example HIV database having over 15,000 strains). By supporting constant-time lookups, the hash table structure dramatically reduces the computational cost and accelerates the overall subtyping process, making it feasible to process large, redundant databases and deliver rapid, high-resolution results for virus subtyping. This capability enables the workflow in process 500 to remain scalable and robust as the number of viral strains and the volume of sequence data continue to grow.

To ensure ongoing accuracy and adaptability, the reference sets can be updated periodically as new genomes are added to the virus data store or database or as subtyping schemes are revised. For example, the process 500 may use a scheduler to update the reference quarterly, semi-annually, or annually. These reference sets can be indexed and stored within the virus data store in a way that supports high-throughput workflows and allows a suitable subtyping system to scale as the number of reference strains and volume of sequence data increase. This comprehensive and flexible approach to reference set generation supports robust, rapid, and high-resolution virus subtyping for a wide range of input data and viral species.

In some embodiments, the process 500 may access a pre-built virus database that already includes reference sets or reference bubbles for each strain, avoiding the need to regenerate these sets at runtime. By accessing a pre-built virus database containing precomputed reference bubbles, the process 500 can rapidly proceed to the subsequent matching and classification steps. The pre-built virus database can also be updated independently as new genomes or subtypes are added, ensuring that the subtyping process remains current without delaying sample analysis.

At block 515, one or more query sequences are obtained for a test sample. The process may include the collection of a biological or environmental sample, which may include blood, plasma, serum, nasopharyngeal swabs, oral swabs, tissue biopsies, cell culture supernatants, or environmental samples containing viral nucleic acids. The genetic material extracted from these samples can be either single-stranded RNA, single-stranded DNA, or double-stranded DNA, depending on the viral species being analyzed. Following extraction, the nucleic acids are subjected to sequencing using an appropriate sequencing technique. Techniques may include high-throughput methods that generate short or long reads, such as next-generation sequencing or nanopore sequencing, or traditional capillary electrophoresis sequencing (e.g., Sanger sequencing) for applications that require longer or more accurate reads.

The sequencing output can be provided as either raw reads or assembled consensus sequences. Raw sequencing reads may be produced as single-end reads, where a sequence is read from one end of a nucleic acid fragment, or as paired-end reads, where sequences are read from both ends. These are usually stored in the FASTQ file format, which encodes both nucleotide sequence and quality scores. In some embodiments, multiple sequencing reads from the test sample are computationally assembled or processed to generate a consensus sequence, which is generally provided in FASTA format. A “query sequence” in this context refers to any nucleotide sequence obtained from the test sample that is analyzed for the purpose of subtype classification, whether it is a single read, a set of reads, or an assembled consensus.

The selection between using a single consensus sequence and multiple raw reads as query sequences depends on the complexity of the viral population and the goals of the analysis. A single consensus sequence may be preferred when the test sample is expected to contain a predominant viral genotype, providing a summary of the most common nucleotide at each position. This is suitable for homogeneous samples or when sequencing depth is limited. In cases where the test sample may contain mixed infections, high intra-host diversity, or minority variants, such as with RNA viruses that exist as quasi-species, multiple raw sequencing reads can be used as separate query sequences to provide a more granular analysis and higher resolution.

Prior to further processing, quality control procedures may be applied to the query sequences to ensure that only high-quality and reliable data are used for subtype classification. These procedures may involve several steps. Raw sequencing reads may be evaluated for overall read quality, and low-quality reads, such as those with average base quality scores below a specified threshold, are filtered out. Sequencing adapters and technical artifacts may be trimmed from the ends of reads to prevent interference with downstream analysis. Reads or consensus sequences that contain ambiguous nucleotides, such as “N” or stretches like “NNN,” are identified and may be removed. For example, if a sequencing read contains a segment like “AGCTNNNCGTAC,” this read may be excluded or the ambiguous region may be trimmed, depending on the pipeline configuration. The same applies to consensus sequences that have ambiguous bases resulting from unresolved positions during assembly.

Sequences that do not meet minimum length requirements (e.g., sequences shorter than the selected k-mer length) may be discarded. Incomplete sequences or truncated records, such as those missing significant portions of the genome or target region, are also excluded. Sequences with excessive ambiguity, such as those with a high proportion of ambiguous bases relative to their total length, are filtered out to maintain the integrity and reliability of the analysis.

Quality control may include the identification and removal of chimeric sequences, which can arise during amplification or sequencing and do not accurately represent the viral population in the sample. Reads or consensus sequences that are found to be contaminated with host genomic material or originate from non-target organisms are also excluded. For example, if a read aligns predominantly to a human reference genome or to a known laboratory contaminant, it is removed from the dataset.

At block 520, each query sequence that has passed quality control is processed to generate a test set of hashed k-mers for the test sample. To create these test sets, each query sequence is divided into overlapping k-mers of the same length k as used for the reference sets. For example, the first k-mer would consist of nucleotides 1 through 15 of the query sequence, the second k-mer would consist of nucleotides 2 through 16, and this pattern continues along the entire length of the query sequence, moving one or more nucleotides at a time. Once all k-mers are extracted from the query sequence, each k-mer is converted into a digital representation by applying the same hash function used to generate the reference sets in the virus data store. For example, if one extracted k-mer is “AGCTTAGCTTAGCTT,” applying the SHA-256 hash function to this k-mer would produce a unique hexadecimal string, which serves as the digital identifier for that k-mer. In some embodiments, the resulting set of hashed k-mers for each query sequence is compiled as the test set. In some embodiments, multiple test sets of the hashed k-mers are generated, each one corresponding to one or more of the query sequences.

In some embodiments, additional quality control or filtering may be applied to the test set after hashing. For example, k-mers with low sequence complexity, repetitive content, or ambiguous bases may be excluded to improve the specificity and reliability of the subtyping process. The final test sets, containing the hashed k-mers derived from quality-controlled query sequences, are then ready for comparison with the reference sets in the next step of process 500.

At block 525, the one or more test sets of hashed k-mers generated from the query sequences are matched with each reference set from the virus data store to generate a k-mer matching score. This k-mer matching score provides a quantitative measure of similarity between the test sample and each reference strain by evaluating how many of the hashed k-mers from the test set are present in each reference set. For example, if a test set contains 1,000 unique hashed k-mers and reference set R1 contains 900 of these hashed k-mers, then the k-mer matching score for reference set R1 would indicate that 900 out of 1,000, or 90 percent of identity. Reference set R2 might contain 700 of these k-mers for a score representing 70 percent, and reference set R3 might only contain 300, for a score representing 30 percent.

In some embodiments, the k-mer matching score includes a penalty for unmatched k-mers to increase the discriminatory power of the scoring system. For example, a scoring algorithm may assign a positive weight equal to the k-mer length (such as 15) for each matching k-mer, while subtracting a penalty value, such as −1, for each unmatched k-mer. This weighted approach ensures that sequences with both a high number of matches and few mismatches will achieve the highest scores, while those with many unmatched k-mers will be penalized, helping to distinguish closely related but non-identical strains. In addition, the matching score may be normalized by the total number of k-mers in the test set or reference set, or expressed as a percentage, to enable fair comparison between samples of different lengths or coverage. In some embodiments, k-mers that are unique to a specific subtype or highly informative for distinguishing between closely related strains may contribute more to the matching score, while common or repetitive k-mers may be down-weighted or excluded from consideration.

In some embodiments, the k-mer matching score is determined using a predetermined algorithm or rule. For example, Equation 1 shows an exemplary formula to generate the k-mer matching score.

$$S = \sum \{ k \vert h ∈ {H}_{r} and h ⊆ {H}_{q} \} + \sum \{ - 1 \vert h ∉ {H}_{r} and h ⊆ {H}_{q} \} \qquad ( Equation 1 )$$

S is the k-mer matching score, k is the length of the k-mers (e.g., k=15), h is a hashed k-mers in the test set, Hr is the reference set of hashed k-mers, and Hq is the test set of hashed k-mers generated from a query read q. When using Equation 1 to determine the k-mer matching score for the test set containing 1,000 unique hashed k-mers and the reference set R1 containing 900 of these hashed k-mers, the k-mer matching score for R1 would be 900*15-100=13,400. Similarly, the k-mer matching score for R2 would be 10,200, and the k-mer matching score for R3 would be 3,800.

The k-mer matching disclosed herein is performed in a position-independent manner, meaning that a match is counted whenever a hashed k-mer is present in both the test set and a reference set, regardless of the original genomic location of the k-mer in either sequence. This approach allows for the detection of shared sequence content even if the query sequence is a partial genome, contains rearranged regions, or has undergone recombination.

The k-mer matching process can be performed in parallel across all reference sets in the virus data store, which increases processing speed and enables the comparison of thousands of query k-mers against many reference strains simultaneously. For example, for a full-length HIV genome of approximately 9,500 nucleotides and a k-mer length of 15, a test sample would generate 9,486 k-mers. In a conventional alignment-based workflow, comparing these k-mers to 15,000 reference genomes of similar size would require 142,290,000 direct sequence comparisons, and if each comparison involved scanning the full genome, this could result in more than a trillion base-to-base checks. By contrast, the disclosed k-mer hashing approach allows each of the 9,486 k-mers to be checked across 15,000 reference sets using hash tables, reducing each membership check to constant time (O(1)). This means only 142,290,000 rapid hash table lookups are needed, compared to more than a trillion base-to-base comparisons required in a conventional alignment-based workflow. This represents a reduction in computational steps and time by a factor of approximately 9,500. This efficiency makes high-throughput, genome-wide subtyping feasible for large and redundant databases, enabling rapid and scalable analysis for research and clinical applications.

At block 530, a query identity for the test sample is determined for each reference set based on the matched k-mers corresponding to the reference set. A query identity for the test sample may be determined for each reference strain in the virus data store, or for the strains with the top k-mer matching score (e.g., top 5%, top 10%, top 20%, or top 50%). Query identity can be calculated as the percentage of bases in the reference genome that are covered by k-mers from the test sample which have matching hashed values in the corresponding reference set. This calculation begins by identifying all k-mers from the test sample that are present in a given reference set and determining the genomic coordinates in the reference genome that these k-mers cover. For example, if the set of matched k-mers collectively spans 8,000 bases in a reference genome of 10,000 bases, the query identity for that reference would be 80 percent. The query identity represents regions of the reference genome with direct sequence support from the test sample, reducing the impact of partial matches or spurious alignments. Because each k-mer represents a continuous stretch of nucleotides, the overall coverage may be calculated by accounting for overlapping k-mers and summing the unique positions they cover in the reference genome. In practice, this query identity metric is robust to gaps, insertions, deletions, and genome rearrangements, since it does not depend on the order or position of k-mers in the query. In some embodiments, if none of strain candidates have at least 80% query identity with the test sample, the subtype will be declared “Undetermined.”

In some embodiments, a consensus sequence of the test sample is generated for each reference set (i.e., different consensus sequences are generated for the test sample). To generate a consensus sequence and determine the query identity using k-mer matching scores, the process begins by matching each hashed k-mer from the query sequence or collection of reads to the reference sets in the virus data store. For each reference strain, the k-mer matching scores are tallied by counting the number of k-mers in the query that are present in the reference set. In some workflows, particularly when processing multiple sequencing reads (such as in FASTQ format), a consensus sequence is constructed by aggregating the results of k-mer matches across all reads. At each nucleotide position in the reference genome, the consensus base is determined by a majority vote of the aligned k-mers that cover that position. This process involves aligning the matched k-mers to their corresponding positions on the reference genome and, at each position, selecting the nucleotide that is most frequently represented among the overlapping k-mers. The resulting consensus sequence represents the predominant genetic composition of the test sample as supported by the k-mer evidence. Once the consensus sequence is assembled, the query identity is calculated as the percentage of bases in the reference genome that are covered by the consensus sequence derived from matched k-mers. This involves identifying the unique positions in the reference genome that are spanned by at least one matched k-mer and dividing the total number of covered bases by the length of the reference genome.

For example, suppose a test sample comprises sequencing reads that are matched to a reference strain of HIV with a genome length of 9,500 bases, using k=15. After matching, it is determined that the matched k-mers from the test sample collectively cover 8,500 unique positions in the reference genome. At each position, the consensus base is selected based on the most common nucleotide among the overlapping k-mers from the test reads. The consensus sequence, therefore, represents the most likely sequence of the test sample as reconstructed from the k-mer matches. The query identity for this reference strain would then be calculated as 8,500 covered bases/9,500 total bases×100%=89.5%. This query identity value indicates that 89.5 percent of the reference genome is covered by the consensus sequence constructed from the k-mer matches. If a threshold is set at 80 percent query identity, this strain would be retained as a candidate for subtype assignment. If multiple reference strains exceed the threshold, their respective query identities are used in the decision rules for final subtype determination, such as consensus assignment or weighted voting.

Query identity serves as an objective, quantitative measure of how closely the test sample resembles each candidate reference strain. This feature can be used as a criterion for filtering candidate strains. In some embodiments, only strains with a query identity above a certain threshold (e.g., at least 80 percent identity) are retained for further consideration. This filtering step reduces false positives and ensures that only the most relevant reference strains are considered for subtype assignment.

In some embodiments, when the analysis of the test sample fails to yield any reference strains with a query identity above the predetermined threshold (for example, 80%), the subtype for the test sample will be reported as “Undetermined.” This outcome indicates that the available sequence data does not provide sufficient evidence to confidently assign the test sample to any known subtype in the reference data store. Such a result may occur when the input sequence is too short, contains excessive ambiguity, or represents a novel or highly divergent strain not present in the current reference set. Reporting an “Undetermined” outcome ensures transparency in cases of insufficient data or ambiguous matches and allows users to pursue additional sequencing, quality control, or database updates as needed

At block 535, an assigned subtype is determined for the test sample based on the k-mer matching scores and/or the query identities. The assignment of subtype may follow a set of decision rules designed to ensure both accuracy and confidence in the classification. In some embodiments, after reference strains in the virus data store are filtered to retain only those with a query identity above a predetermined threshold (e.g., ≥80%), a determination is made whether the top candidate strains with the highest query identities (e.g., the top four candidates) all share the same subtype. If there is agreement among these top strains, the same subtype is directly assigned to the test sample with a “higher” confidence. For example, if the top four candidate strains with the highest query identities are all subtype B, then subtype B is assigned as the result with a “higher” confidence. In some embodiments, the determination is based on the k-mer matching scores.

If the top candidate strains do not unanimously agree on the same subtype, a weighted voting procedure may be applied. In this approach, each candidate subtype is assigned a weight that is proportional to the sum of the query identities of all strains within that subtype. The weight may also reflect the k-mer matching score (e.g., using it as a weight for each query identity). For example, if a test sample's top four candidates include two strains of subtype C (with query identities of 85 percent and 83 percent) and two strains of subtype G (with query identities of 82 percent and 80 percent), the weights for subtypes C and G can be 168 and 162, respectively. The subtype with the highest aggregated weight, in this case subtype C, is selected as the assigned subtype, reflecting a majority consensus among the closest matches. Additionally, the subtype with the second highest weight may be reported as a secondary or supplementary classification to inform users of possible ambiguity or mixtures in the test sample. For example, if a sample contains a mixture of subtypes or represents a recombinant, reporting the secondary subtype provides valuable context for interpretation. In some embodiments, the assigned subtype is one or more specific subtype from the plurality of subtypes of the virus of interest, or the assigned subtype is marked as “undetermined.”

This combination of thresholding, direct assignment, and weighted voting enables the workflow to deliver confident and reproducible subtyping results even in challenging scenarios, such as mixed infections, recombinants, or cases with limited sequence coverage. For example, when analyzing a sample that contains both subtype A and subtype G variants, the process may indicate a primary assignment of subtype A with a “high” confidence, and also report subtype G as a secondary result if it is significantly represented among the top candidates. This systematic approach minimizes misclassification and provides clear, interpretable results for a wide range of clinical and research applications, supporting robust virus subtyping even in complex and ambiguous cases. Different categorical confidence levels or numerical confidence levels may be assigned.

A range of technical and practical operations can be integrated into the process 500 after the subtype is assigned, optionally automated within a laboratory information management system (LIMS) or a clinical data infrastructure, ensuring that the assigned subtype, k-mer matching scores, query identity, and relevant metadata are formatted into structured digital reports. For example, upon subtyping an HCV sample, the results may be automatically formatted as an HL7 or XML report, which is then uploaded to a hospital's LIMS and flagged for review by a clinical virologist. This report could include supporting evidence such as genome coverage plots, consensus sequence data, and quality metrics, ensuring traceability and compliance with regulatory standards.

The assigned subtype can also serve as a trigger for downstream computational analyses. In clinical applications, the subtype result may initiate drug resistance profiling by cross-referencing the identified subtype with a curated resistance mutation database, generating a personalized antiviral treatment recommendation. In public health surveillance, subtype assignments can be aggregated and mapped to monitor the spread and emergence of specific viral subtypes or recombinant forms. For example, identification of a novel recombinant HIV subtype in a cohort of patient samples could trigger a public health alert and initiate further epidemiological investigations.

Laboratory workflows may further utilize subtype assignments to inform reflex testing protocols or sample archiving. For example, detection of a rare or recombinant subtype in a blood donor sample can automatically prompt confirmatory sequencing of additional genomic regions. Subtype results can also be used to prioritize samples for long-term biobanking or for inclusion in ongoing research studies on viral diversity.

Visualization tools and interactive dashboards can display the assigned subtype alongside supporting metrics, allowing laboratory scientists, bioinformaticians, or clinicians to review detailed evidence for subtype assignment. These interfaces may present heatmaps of k-mer matching scores, genome coverage graphs, and interactive phylogenetic trees, facilitating manual review, annotation, and decision-making in complex or ambiguous cases. For example, an interactive dashboard could allow a virologist to explore all candidate subtypes with their respective weights and query identities, reviewing cases where mixtures or recombinants are suspected.

Additionally, data generated through the process 500, including raw and processed sequence files, hashed k-mer sets, matching scores, consensus sequences, and assigned subtypes, can be archived in secure, version-controlled databases. This supports future retrieval, audit, reanalysis, or regulatory review, and ensures data integrity and reproducibility. Automated data retention schedules, backup protocols, and controlled access systems help maintain compliance with institutional and legal requirements.

### VII. EXAMPLES

The examples below are intended to further illustrate certain aspects of the techniques described herein and are not intended to limit the scope of the claims.

### Example 1: HIV Subtyping

Human Immunodeficiency Virus (HIV) exhibits extensive genetic diversity, with numerous subtypes and recombinant forms circulating globally. Accurate subtyping is critical for clinical diagnostics, treatment selection, epidemiological surveillance, and vaccine development. The k-mer-based virus subtyping pipeline described here is designed to assign an HIV subtype to a sample based on either consensus sequence data (FASTA format) or raw sequencing reads (FASTQ format), supporting both traditional and next-generation sequencing workflows.

A comprehensive HIV reference database was constructed by aggregating 15,336 complete HIV genomes across 32 subtypes, including A, A1, A2, A3, A4, A6, A7, A8, B, C, D, F1, F2, G, H, J, K, L, N, O, P, U, AE, AG, AB, DF, BC, CD, BF, BG, Complex, and SIV. All sequences were sourced from the LANL HIV sequence database, and the database was curated to ensure completeness and accurate subtype annotation. Only high-quality, full-length genomes with clear subtype labels were retained, and the database is periodically updated to reflect new strain discoveries and nomenclature changes.

Reference bubbles were precomputed for each strain in the database. For each genome, all possible 15-base k-mers were extracted and hashed to create a digital fingerprint unique to that strain. These reference bubbles are stored in hash tables or sets to enable constant-time membership checks and rapid comparison during subtyping analysis.

The pipeline accepts input as either FASTQ files (raw reads) or FASTA files (consensus sequences) from patient samples. If a consensus sequence contains ambiguous bases or “NNN” runs, it is split into subsequences for independent analysis. In the case of FASTQ input, each read is processed individually.

Preprocessing steps include quality filtering to exclude low-quality reads or bases, adapter trimming, and format conversion as needed. These procedures ensure that only reliable, high-quality sequence data are advanced to the k-mer extraction stage.

All possible 15-mers from each input sequence are hashed using the same hash function applied during reference bubble construction. This generates a set of hashed k-mers representing the sample's digital fingerprint. A matching score is computed by assigning a positive weight (15) for each match and a penalty (−1) for each unmatched k-mer. This process can be run in parallel across all reference sets. A consensus sequence may be generated from the aggregation of k-mer matches, and a query identity metric is calculated as the percentage of bases in the reference genome covered by matching k-mers. Only reference strains with a query identity above a set threshold (80%) are considered for final subtype assignment

Up to four top candidate strains with the highest query identities are selected. The two-condition decision rule was applied to identify subtypes and confidence levels.

The k-mer-based HIV subtyping pipeline was evaluated on a range of datasets, including four HIV POL sample sets, 36 non-B samples, 40 samples covering 20 subtypes, 73 complex and undetermined samples, and 2,369 production samples. Perfect concordance (100%) was achieved with known subtypes for 36 non-B samples. High-confidence subtypes were called for all complex and undetermined samples, with k-mer subtyping matching BLAST results, with 96.5% concordance between k-mer subtyping and the existing inhouse pipeline across 2,369 production samples and for resolution of complex and undetermined cases, with k-mer subtyping correctly identifying challenging samples in 89% of discordant cases, compared to 11% for the existing conventional method.

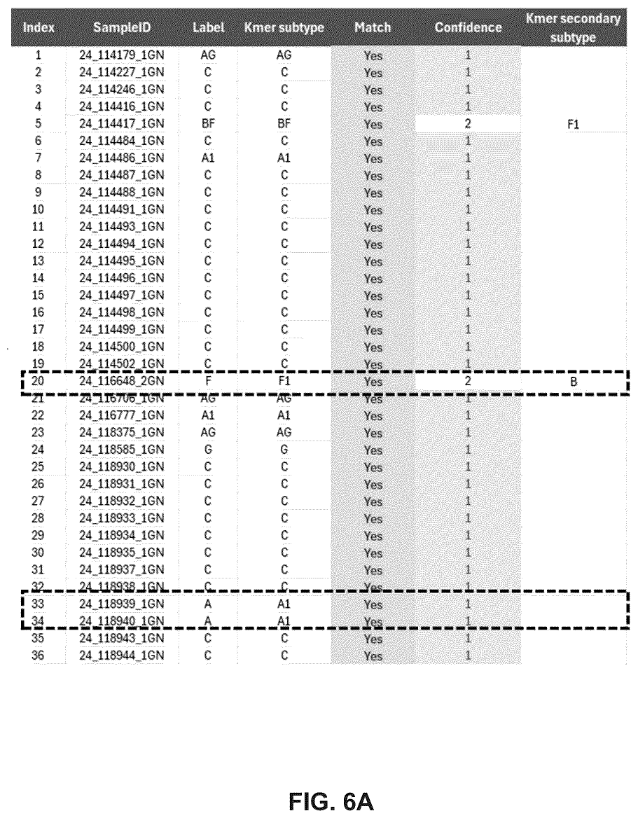

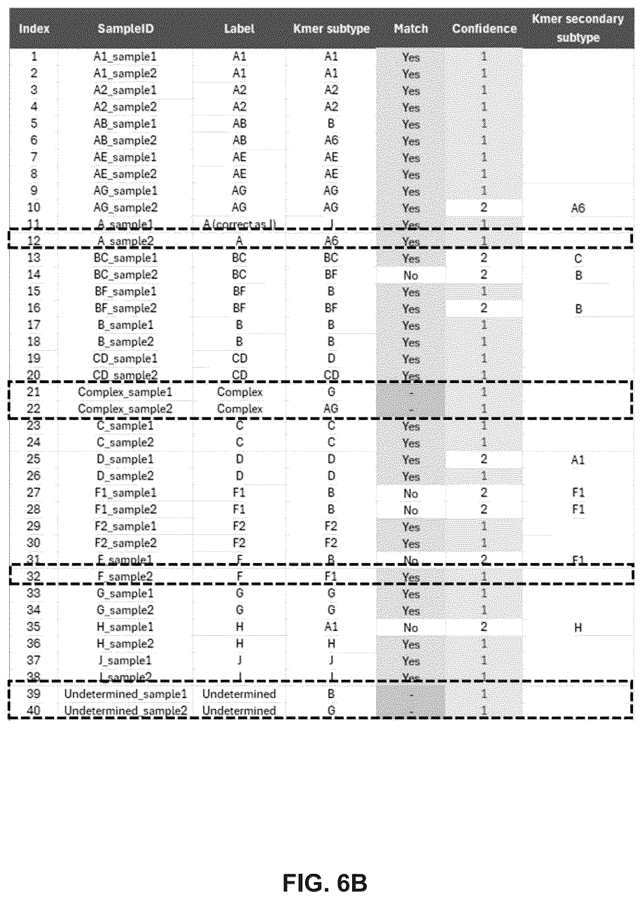

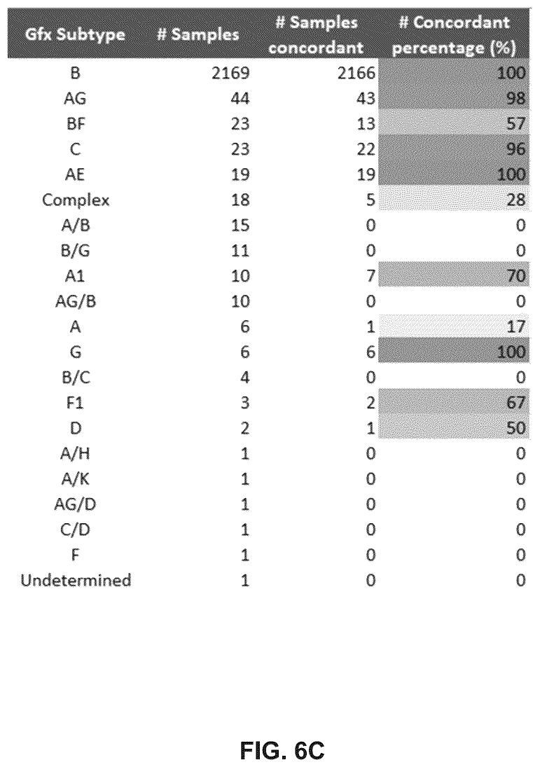

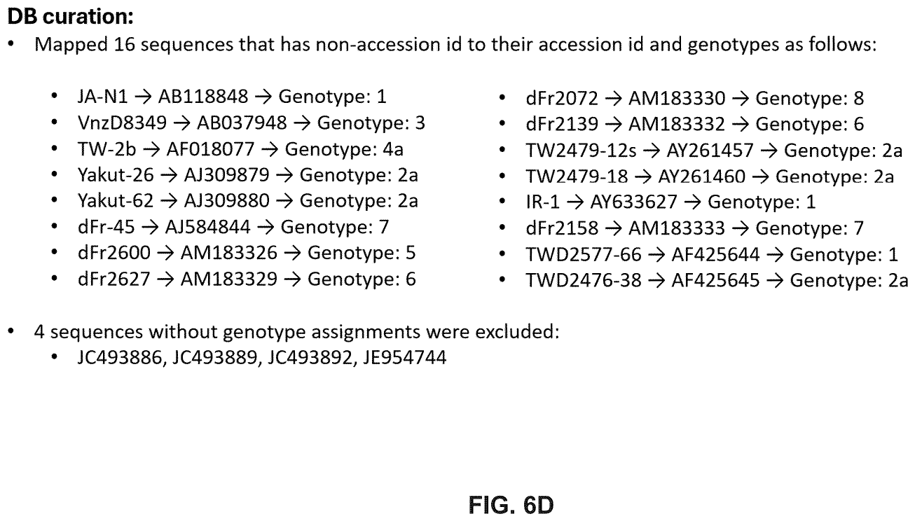

For example, FIG. 6A shows for all (36 of 36, 100%) non-B samples, the k-mer-based subtyping calls matched the provided labels and that k-mer subtyping techniques can provide subtype calls with higher resolution (e.g., on samples #33 and 34: A v.s. A1). FIG. 6B shows

For samples with ambiguous or low-identity consensus sequences, such as those with high percentages of “N” bases, the pipeline can process the raw FASTQ reads to improve subtype determination.

FIG. 6B shows a comparative table summarizing the performance evaluation of the k-mer-based HIV subtyping pipeline across multiple test samples. The table indicates that for 40 samples representing 20 subtype labels, 87.5% showed concordant results. The table also includes columns for additional metrics such as the number of complex or undetermined cases resolved, highlighting scenarios where the k-mer-based pipeline provided higher-resolution subtyping or clarified ambiguous results. All reported values in FIG. 6B, including sample counts, concordance rates, and subtype classifications, are exemplary and intended to illustrate the performance characteristics of the system. These metrics should not be construed as limiting and may vary with different datasets, populations, or future updates to the reference database or workflow parameters.

FIG. 6C is a table illustrating the concordance analysis of 2,369 HIV samples. The distribution of HIV samples are across various subtypes and the concordance percentage for each subtype. Among them, 2,285 of 2,369 samples (96.5%) had concordant subtyping calls via conventional subtyping and k-mer subtyping, subtypes B, AG, C, AE, and G each had at least 96% concordance between subtyping methods, 84 samples had discordant subtyping calls, in which 4 are duplicate samples, and 80 are unique samples which are mainly non-B subtypes, such as mixture samples, complex samples, and subtype A samples.

### Example 2: HCV Subtyping

Hepatitis C Virus (HCV) is a highly diverse RNA virus with over 100 subtypes spanning multiple genotypes. Accurate HCV subtyping is essential for clinical management, resistance testing, surveillance, and research. The k-mer-based virus subtyping pipeline described here is adapted for HCV to address the challenges of high sequence variability and the occurrence of inter-subtype recombinants. The system supports input from both consensus sequences (FASTA) and raw sequencing reads (FASTQ) and can be applied to data from any region of the HCV genome, enabling broad applicability in clinical and research environments. A similar subtyping pipeline was used to process the HCV data (3,767 complete genomes with annotated subtype labels (112 subtypes) were downloaded from the LANL HCV sequence database). A chunk module was added to the pipeline (see FIG. 3).

During curation of the HCV reference database, a comprehensive quality review and harmonization process was performed to ensure consistency and accuracy in subtype labeling. This included relabeling the LANL subtype for 95 sequences: 86 sequences were relabeled to resolve differences in case formatting (for example, converting 1A to 1a), 6 sequences with ambiguous labels were clarified, and 3 recombinant sequences were updated to match convention for recombinant labeling. In addition to these corrections, 28 new sequences with confirmed subtypes were added to the database. This expansion introduced 14 new subtypes, including subtypes 1d, 1i, 1j, 1k, 1m, 1n, 1o, 3d, 3e, 6xb, 6xd, 6xe, 6xh, and 7b, as recognized by ICTV (International Committee on Taxonomy of Viruses). The curation process also ensured that all sequences included in the FDA's recommended list of HCV references for resistance data submission were present in the database. This rigorous curation resulted in a high-quality, comprehensive HCV reference database, supporting reliable and robust subtyping analysis for clinical and research applications.

The subtyping pipeline is evaluated on two datasets: NS5B (101 full-length sequence samples, and Sanger sequencing (2,969 in silico amplicons from LANL sequences. Each dataset was analyzed using the environment and workflows described in FIGS. 2-5, with results demonstrating high concordance with known subtypes, accurate detection of recombinants, and effective handling of diverse sample types and sequencing modalities.

For the NS5B dataset, the k-mer-based subtyping pipeline was evaluated alongside existing subtyping methods. The results demonstrated that the k-mer-based pipeline and the existing subtyping methods produced concordant subtyping calls for nearly all samples in this dataset. There was only one discordant sample (GT2a-80), where the existing method assigned subtype 2a, while the k-mer-based method assigned subtype 2k. The top BLAST result for this sample also matched the k-mer-based subtype call of 2k. Additionally, the k-mer-based pipeline provided higher-resolution subtype assignments for 22 samples within the NS5B dataset. These findings highlight the accuracy, specificity, and added resolution of the k-mer-based method for standard HCV genotyping tasks, supporting its application in clinical and research environments.

For the HCV Sanger Sequencing dataset, HCV genotype sequencing is performed via nested PCR followed by Sanger, with NS5B being the target gene for the PCR design. Combined outer/inner in silico PCR results with at most 5 primer mismatches produces 2,969 in silico Sanger amplicons. The k-mer-based subtyping pipeline was evaluated using 2,969 in silico amplicons. The results showed that 2,949 out of 2,969 amplicons (99.3%) had k-mer-based subtype calls that matched the subtype labels provided in the LANL database. The 20 remaining amplicons (0.7%) that showed mismatches were found to be identical across multiple subtypes, making them inherently indistinguishable by any subtyping method. In these cases, when an input sequence was found in multiple reference subtypes, the k-mer-based pipeline assigned the subtype with the highest percentage of matching k-mers, a selection that sometimes favored shorter reference sequences with fewer total k-mers, which may belong to a different subtype. This led to the observed mismatches. Overall, the k-mer-based pipeline achieved perfect subtyping accuracy for Sanger in silico amplicons within the limits of sequence distinguishability, demonstrating its reliability and precision for HCV genotyping tasks in both simulated and practical laboratory contexts.

### Example 3: HDV Subtyping

Hepatitis Delta Virus (HDV) is a clinically important RNA virus with eight main genotypes, each associated with distinct geographic distributions and clinical outcomes. Accurate HDV genotyping supports clinical management, epidemiological surveillance, and research into viral evolution. The k-mer-based virus subtyping pipeline described here is adapted for HDV genotyping and designed to assign the correct genotype based on either consensus sequence data (FASTA format) or raw sequencing reads (FASTQ format). The system can process input from any region of the HDV genome, making it broadly applicable in both research and diagnostic settings.

The HDV reference database was built by downloading 220 complete genomes from the vHDvDB 2.0 resource. During curation, 16 sequences with non-accession IDs were carefully mapped to their accession IDs and genotypes, ensuring that each genome was accurately represented in the reference panel. Four sequences lacking genotype assignments were excluded to maintain the integrity and specificity of the database. The final curated KmerHDV reference database contains 216 sequences representing eight main genotypes. FIG. 6D illustrates the data curation process.

The HDV genotyping pipeline was evaluated on multiple sample sets. In a test of ten samples (see Table 1 below), all eight samples with known genotype labels were correctly genotyped by the k-mer-based method, and for two samples without prior labels, the pipeline provided high-confidence predictions. In an additional set of nine samples (see Table 2 below), seven of eight labeled samples showed concordance between k-mer-based predictions and known labels. One sample (HDV13) displayed a minor discrepancy, which was traced to a database genotype label issue rather than a misclassification. Unlabeled samples were also successfully genotyped with high confidence using the pipeline. Overall, the k-mer-based HDV genotyping system demonstrated high accuracy and reliability, even for challenging or ambiguous cases.

TABLE 1

| | | Kmer | Kmer Closest |
|---|---|---|---|
| Sample Name | Sample Target | Genotype | Accession |
| HDV01 | Patient Sample (unknown genotype) | 1 | AM902166 |
| HDV02 | HDV 8a_AJ584849 | 8 | AJ584849 |
| HDV03 | HDV 7a_AJ584844 | 7 | AJ584844 |
| HDV04 | HDV 6b_JX888102 | 6 | JX888102 |
| HDV05 | HDV 5a_JX888103 | 5 | JX888103 |
| HDV06 | HDV 4a_AF018077 | 4a | AF018077 |
| HDV07 | HDV 3b_AB037947 | 3 | AB037947 |
| HDV08 | HDV 2a_X60193 | 2a | AB118846 |
| HDV09 | HDV 1a_JX888100 | 1 | JX888100 |
| HDV10 | HDV Ref_NC_001653.2 | 1 | D01075 |

TABLE 2

| | | Kmer | Kmer Closest |
|---|---|---|---|
| Sample Name | Sample Target | Genotype | Accession |
| HDV11 | Patient Sample | 1 | AM902166 |
| HDV12 | HDV 1b_JX888098 | 1 | JX888098 |
| HDV13 | HDV 2b_AJ309879 | 2a | AJ309879 |
| HDV14 | HDV 3c_KC590319 | 3 | KC590319 |
| HDV15 | HDV 4b_AB118818 | 4b | AB118818 |
| HDV16 | HDV 5b_AM183331 | 5 | AM183331 |
| HDV17 | HDV 6a_AJ584847 | 6 | AJ584847 |
| HDV18 | HDV 7b_AM183333 | 7 | AM183333 |
| HDV19 | HDV 8b_LT594488 | 8 | AM183330 |

The results presented in Examples 1 through 3 demonstrate that the k-mer-based viral subtyping pipeline is both fast and accurate across a spectrum of clinically relevant viruses, including HIV, HCV, and HDV. For HIV, the pipeline achieved high concordance with known subtypes, resolved complex and ambiguous cases that challenged previous methods, and delivered high-resolution results within seconds, supporting both consensus sequence and raw sequencing read inputs. For HCV, the pipeline assigned subtypes accurately and detected recombinants across large and diverse datasets, with performance validated on full-length genomes, in silico Sanger amplicons, and challenging recombinant samples; the inclusion of a chunking module further increased sensitivity for mosaic genomes. In HDV genotyping, the pipeline correctly classified all evaluated samples, maintained high accuracy in ambiguous situations, and was compatible with a range of sequencing technologies and laboratory workflows.

In addition to these empirical results, the k-mer-based viral subtyping pipeline has four principal advantages: it is automated, fast and accurate, adaptable, and broadly applicable. First, automation is provided at two levels. The k-mer hashing databases are pre-built and self-included, and can be readily updated as new complete viral genomes or subtypes become publicly validated and available on a quarterly, semi-annual, or annual cadence, and subtyping itself is fully automated from a single command line that accepts both FASTA and FASTQ inputs. Second, relative to the previously used conventional bioinformatic subtyping approach that primarily relies on BLASTn comparisons to a reference database, the k-mer-based method is significantly faster and provides higher-resolution calls, delivering accurate results within seconds even for mixed or complex samples. Third, the pipeline is adaptable across genomic regions because the k-mer hashing database is constructed from complete genomes, enabling subtyping on pol, env, gag, or other regions, as well as whole genomes. Fourth, the modular design and flexible architecture extend beyond HIV, HCV, and HDV, allowing straightforward adaptation to additional viruses, which enhances its utility for high-throughput clinical diagnostics, surveillance, and research

### VIII. MACHINES, SOFTWARE AND INTERFACES

Certain processes and methods described herein (obtaining, inquiring, generating, identifying, curating, creating, testing, and the like) are often performed with a computer, microprocessor, software, subsystem or other machine. Methods described herein typically are computer-implemented methods, and one or more portions of a method sometimes are performed by one or more processors (e.g., microprocessors), computers, systems, apparatuses, or machines (e.g., microprocessor-controlled machine).

Computers, systems, apparatuses, machines and computer program products suitable for use often include, or are utilized in conjunction with, computer readable storage media. Non-limiting examples of computer readable storage media include memory, hard disk, CD-ROM, flash memory device and the like. Computer readable storage media generally are computer hardware, and often are non-transitory computer-readable storage media. Computer readable storage media are not computer readable transmission media, the latter of which are transmission signals per se.

Machines, software and interfaces may be used to conduct methods described herein. Using machines, software and interfaces, a user may enter, request, query or determine options for using particular information, programs or processes (e.g., mapping sequence reads, processing mapped data and/or providing an outcome), which can involve implementing statistical analysis algorithms, statistical significance algorithms, statistical algorithms, iterative steps, validation algorithms, and graphical representations, for example. In some embodiments, a data set may be entered by a user as input information, a user may download one or more data sets by suitable hardware media (e.g., flash drive), and/or a user may send a data set from one system to another for subsequent processing and/or providing an outcome (e.g., send sequence read data from a sequencer to a computer system for sequence read mapping; send mapped sequence data to a computer system for processing and yielding an outcome and/or report).

A system typically comprises one or more machines. Each machine comprises one or more of memory, one or more microprocessors, and instructions. Where a system includes two or more machines, some or all of the machines may be located at the same location, some or all of the machines may be located at different locations, all of the machines may be located at one location and/or all of the machines may be located at different locations. Where a system includes two or more machines, some or all of the machines may be located at the same location as a user, some or all of the machines may be located at a location different than a user, all of the machines may be located at the same location as the user, and/or all of the machine may be located at one or more locations different than the user.

A system sometimes comprises a computing machine and a sequencing apparatus or machine, where the sequencing apparatus or machine is configured to receive physical nucleic acid and generate sequence reads, and the computing apparatus is configured to process the reads from the sequencing apparatus or machine. The computing machine sometimes is configured to determine a classification outcome from the sequence reads.

A user may, for example, place a query to software which then may acquire a data set via internet access, and in certain embodiments, a programmable microprocessor may be prompted to acquire a suitable data set based on given parameters. A programmable microprocessor also may prompt a user to select one or more data set options selected by the microprocessor based on given parameters. A programmable microprocessor may prompt a user to select one or more data set options selected by the microprocessor based on information found via the internet, other internal or external information, or the like. Options may be chosen for selecting one or more data feature selections, one or more statistical algorithms, one or more statistical analysis algorithms, one or more statistical significance algorithms, iterative steps, one or more validation algorithms, and one or more graphical representations of methods, machines, apparatuses, computer programs or a non-transitory computer-readable storage medium with an executable program stored thereon.

Systems addressed herein may comprise general components of computer systems, such as, for example, network servers, laptop systems, desktop systems, handheld systems, personal digital assistants, computing kiosks, and the like. A computer system may comprise one or more input means such as a keyboard, touch screen, mouse, voice recognition or other means to allow the user to enter data into the system. A system may further comprise one or more outputs, including, but not limited to, a display screen (e.g., CRT or LCD), speaker, FAX machine, printer (e.g., laser, ink jet, impact, black and white or color printer), or other output useful for providing visual, auditory and/or hardcopy output of information (e.g., outcome and/or report).

In a system, input and output components may be connected to a central processing unit which may comprise among other components, a microprocessor for executing program instructions and memory for storing program code and data. In some embodiments, processes may be implemented as a single user system located in a single geographical site. In certain embodiments, processes may be implemented as a multi-user system. In the case of a multi-user implementation, multiple central processing units may be connected by means of a network. The network may be local, encompassing a single department in one portion of a building, an entire building, span multiple buildings, span a region, span an entire country or be worldwide. The network may be private, being owned and controlled by a provider, or it may be implemented as an internet-based service where the user accesses a web page to enter and retrieve information. Accordingly, in certain embodiments, a system includes one or more machines, which may be local or remote with respect to a user. More than one machine in one location or multiple locations may be accessed by a user, and data may be mapped and/or processed in series and/or in parallel. Thus, a suitable configuration and control may be utilized for mapping and/or processing data using multiple machines, such as in local network, remote network and/or “cloud” computing platforms.

A system can include a communications interface in some embodiments. A communications interface allows for transfer of software and data between a computer system and one or more external devices. Non-limiting examples of communications interfaces include a modem, a network interface (such as an Ethernet card), a communications port, a PCMCIA slot and card, and the like. Software and data transferred via a communications interface generally are in the form of signals, which can be electronic, electromagnetic, optical and/or other signals capable of being received by a communications interface. Signals often are provided to a communications interface via a channel. A channel often carries signals and can be implemented using wire or cable, fiber optics, a phone line, a cellular phone link, an RF link and/or other communications channels. Thus, in an example, a communications interface may be used to receive signal information that can be detected by a signal detection subsystem.

Data may be input by a suitable device and/or method, including, but not limited to, manual input devices or direct data entry devices (DDEs). Non-limiting examples of manual devices include keyboards, concept keyboards, touch sensitive screens, light pens, mouse, tracker balls, joysticks, graphic tablets, scanners, digital cameras, video digitizers and voice recognition devices. Non-limiting examples of DDEs include bar code readers, magnetic strip codes, smart cards, magnetic ink character recognition, optical character recognition, optical mark recognition, and turnaround documents.

In some embodiments, output from a sequencing apparatus or machine may serve as data that can be input via an input device. In certain embodiments, mapped sequence reads may serve as data that can be input via an input device. In certain embodiments, nucleic acid fragment size (e.g., length) may serve as data that can be input via an input device. In certain embodiments, output from a nucleic acid capture process (e.g., genomic region origin data) may serve as data that can be input via an input device. In certain embodiments, a combination of nucleic acid fragment size (e.g., length) and output from a nucleic acid capture process (e.g., genomic region origin data) may serve as data that can be input via an input device. In certain embodiments, simulated data is generated by an in silico process and the simulated data serves as data that can be input via an input device. The term “in silico” refers to research and experiments performed using a computer. In silico processes include, but are not limited to, mapping sequence reads and processing mapped sequence reads according to processes described herein.

A system may include software useful for performing a process or part of a process described herein, and software can include one or more subsystems for performing such processes (e.g., sequencing subsystem, logic processing subsystem, data display organization subsystem). The term “software” refers to computer readable program instructions that, when executed by a computer, perform computer operations. Instructions executable by the one or more microprocessors sometimes are provided as executable code, that when executed, can cause one or more microprocessors to implement a method described herein.

A subsystem described herein can exist as software, and instructions (e.g., processes, routines, subroutines) embodied in the software can be implemented or performed by a microprocessor. For example, a subsystem (e.g., a software subsystem) can be a part of a program that performs a particular process or task. The term “subsystem” refers to a self-contained functional unit that can be used in a larger machine or software system. A subsystem can comprise a set of instructions for carrying out a function of the subsystem. A subsystem can transform data and/or information. Data and/or information can be in a suitable form. For example, data and/or information can be digital or analogue. In certain embodiments, data and/or information sometimes can be packets, bytes, characters, or bits. In some embodiments, data and/or information can be any gathered, assembled or usable data or information. Non-limiting examples of data and/or information include a suitable media, pictures, video, sound (e.g. frequencies, audible or non-audible), numbers, constants, a value, objects, time, functions, instructions, maps, references, sequences, reads, mapped reads, levels, ranges, thresholds, signals, displays, representations, or transformations thereof. A subsystem can accept or receive data and/or information, transform the data and/or information into a second form, and provide or transfer the second form to a machine, peripheral, component or another subsystem. A subsystem can perform one or more of the following non-limiting functions: mapping sequence reads, providing counts, assembling portions, providing or determining a level, providing a count profile, normalizing (e.g., normalizing reads, normalizing counts, and the like), providing a normalized count profile or levels of normalized counts, comparing two or more levels, providing uncertainty values, providing or determining expected levels and expected ranges (e.g., expected level ranges, threshold ranges and threshold levels), providing adjustments to levels (e.g., adjusting a first level, adjusting a second level, adjusting a profile of a chromosome or a part thereof, and/or padding), providing identification (e.g., identifying a copy number alteration, genetic variation/genetic alteration or aneuploidy), categorizing, plotting, and/or determining an outcome, for example. A microprocessor can, in certain embodiments, carry out the instructions in a subsystem. In some embodiments, one or more microprocessors are required to carry out instructions in a subsystem or group of subsystems. A subsystem can provide data and/or information to another subsystem, machine or source and can receive data and/or information from another subsystem, machine or source.

A computer program product sometimes is embodied on a tangible computer-readable medium, and sometimes is tangibly embodied on a non-transitory computer-readable medium. A subsystem sometimes is stored on a computer readable medium (e.g., disk, drive) or in memory (e.g., random access memory). A subsystem and microprocessor capable of implementing instructions from a subsystem can be located in a machine or in a different machine. A subsystem and/or microprocessor capable of implementing an instruction for a subsystem can be located in the same location as a user (e.g., local network) or in a different location from a user (e.g., remote network, cloud system). In embodiments in which a method is carried out in conjunction with two or more subsystems, the subsystems can be located in the same machine, one or more subsystems can be located in different machine in the same physical location, and one or more subsystems may be located in different machines in different physical locations.

A machine, in some embodiments, comprises at least one microprocessor for carrying out the instructions in a subsystem. Sequence read quantifications (e.g., counts) sometimes are accessed by a microprocessor that executes instructions configured to carry out a method described herein. Sequence read quantifications that are accessed by a microprocessor can be within memory of a system, and the counts can be accessed and placed into the memory of the system after they are obtained. In some embodiments, a machine includes a microprocessor (e.g., one or more microprocessors) which microprocessor can perform and/or implement one or more instructions (e.g., processes, routines and/or subroutines) from a subsystem. In some embodiments, a machine includes multiple microprocessors, such as microprocessors coordinated and working in parallel. In some embodiments, a machine operates with one or more external microprocessors (e.g., an internal or external network, server, storage device and/or storage network (e.g., a cloud)). In some embodiments, a machine comprises a subsystem (e.g., one or more subsystems). A machine comprising a subsystem often is capable of receiving and transferring one or more of data and/or information to and from other subsystems.

In certain embodiments, a machine comprises peripherals and/or components. In certain embodiments, a machine can comprise one or more peripherals or components that can transfer data and/or information to and from other subsystems, peripherals and/or components. In certain embodiments, a machine interacts with a peripheral and/or component that provides data and/or information. In certain embodiments, peripherals and components assist a machine in carrying out a function or interact directly with a subsystem. Non-limiting examples of peripherals and/or components include a suitable computer peripheral, I/O or storage method or device including but not limited to scanners, printers, displays (e.g., monitors, LED, LCT or CRTs), cameras, microphones, pads (e.g., iPads, tablets), touch screens, smart phones, mobile phones, USB I/O devices, USB mass storage devices, keyboards, a computer mouse, digital pens, modems, hard drives, jump drives, flash drives, a microprocessor, a server, CDs, DVDs, graphic cards, specialized I/O devices (e.g., sequencers, photo cells, photo multiplier tubes, optical readers, sensors, etc.), one or more flow cells, fluid handling components, network interface controllers, ROM, RAM, wireless transfer methods and devices (Bluetooth, WiFi, and the like,), the world wide web (www), the internet, a computer and/or another subsystem.

Software comprising program instructions often is provided on a program product containing program instructions recorded on a computer readable medium, including, but not limited to, magnetic media including floppy disks, hard disks, and magnetic tape; and optical media including CD-ROM discs, DVD discs, magneto-optical discs, flash memory devices (e.g., flash drives), RAM, floppy discs, the like, and other such media on which the program instructions can be recorded. In online implementation, a server and web site maintained by an organization can be configured to provide software downloads to remote users, or remote users may access a remote system maintained by an organization to remotely access software. Software may obtain or receive input information. Software may include a subsystem that specifically obtains or receives data (e.g., a data receiving subsystem that receives sequence read data and/or mapped read data) and may include a subsystem that specifically processes the data (e.g., a processing subsystem that processes received data (e.g., filters, normalizes, provides an outcome and/or report). The terms “obtaining” and “receiving” input information refers to receiving data (e.g., sequence reads, mapped reads) by computer communication means from a local, or remote site, human data entry, or any other method of receiving data. The input information may be generated in the same location at which it is received, or it may be generated in a different location and transmitted to the receiving location. In some embodiments, input information is modified before it is processed (e.g., placed into a format amenable to processing (e.g., tabulated)).

Software can include one or more algorithms in certain embodiments. An algorithm may be used for processing data and/or providing an outcome or report according to a finite sequence of instructions. An algorithm often is a list of defined instructions for completing a task. Starting from an initial state, the instructions may describe a computation that proceeds through a defined series of successive states, eventually terminating in a final ending state. The transition from one state to the next is not necessarily deterministic (e.g., some algorithms incorporate randomness). By way of example, and without limitation, an algorithm can be a search algorithm, sorting algorithm, merge algorithm, numerical algorithm, graph algorithm, string algorithm, modeling algorithm, computational genomic algorithm, combinatorial algorithm, machine learning algorithm, cryptography algorithm, data compression algorithm, parsing algorithm and the like. An algorithm can include one algorithm or two or more algorithms working in combination. An algorithm can be of any suitable complexity class and/or parameterized complexity. An algorithm can be used for calculation and/or data processing, and in some embodiments, can be used in a deterministic or probabilistic/predictive approach. An algorithm can be implemented in a computing environment by use of a suitable programming language, non-limiting examples of which are C, C++, Java, Perl, Python, Fortran, and the like. In some embodiments, an algorithm can be configured or modified to include margin of errors, statistical analysis, statistical significance, and/or comparison to other information or data sets (e.g., applicable when using a neural net or clustering algorithm).

In certain embodiments, several algorithms may be implemented for use in software. These algorithms can be trained with raw data in some embodiments. For each new raw data sample, the trained algorithms may produce a representative processed data set or outcome. A processed data set sometimes is of reduced complexity compared to the parent data set that was processed. Based on a processed set, the performance of a trained algorithm may be assessed based on sensitivity and specificity, in some embodiments. An algorithm with the highest sensitivity and/or specificity may be identified and utilized, in certain embodiments.

In certain embodiments, simulated (or simulation) data can aid data processing, for example, by training an algorithm or testing an algorithm. Simulated data also is referred to herein as “virtual” data. Simulations can be performed by a computer program in certain embodiments. One possible step in using a simulated data set is to evaluate the confidence of identified results. One approach is to calculate a probability value (p-value), which estimates the probability of a random sample having better score than the selected samples. In some embodiments, an empirical model may be assessed, in which it is assumed that at least one sample matches a reference sample (with or without resolved variations). In some embodiments, another distribution, such as a Poisson distribution for example, can be used to define the probability distribution.

A system may include one or more microprocessors in certain embodiments. A microprocessor can be connected to a communication bus. A computer system may include a main memory, often random access memory (RAM), and can also include a secondary memory. Memory in some embodiments comprises a non-transitory computer-readable storage medium. Secondary memory can include, for example, a hard disk drive and/or a removable storage drive, representing a floppy disk drive, a magnetic tape drive, an optical disk drive, memory card and the like. A removable storage drive often reads from and/or writes to a removable storage unit. Non-limiting examples of removable storage units include a floppy disk, magnetic tape, optical disk, and the like, which can be read by and written to by, for example, a removable storage drive. A removable storage unit can include a computer-usable storage medium having stored therein computer software and/or data.

A microprocessor may implement software in a system. In some embodiments, a microprocessor may be programmed to automatically perform a task described herein that a user could perform. Accordingly, a microprocessor, or algorithm conducted by such a microprocessor, can require little to no supervision or input from a user (e.g., software may be programmed to implement a function automatically). In some embodiments, the complexity of a process is so large that a single person or group of persons could not perform the process in a time frame short enough for determining the presence or absence of a genetic variation or genetic alteration.

In some embodiments, secondary memory may include other similar means for allowing computer programs or other instructions to be loaded into a computer system. For example, a system can include a removable storage unit and an interface device. Non-limiting examples of such systems include a program cartridge and cartridge interface (such as that found in video game devices), a removable memory chip (such as an EPROM, or PROM) and associated socket, and other removable storage units and interfaces that allow software and data to be transferred from the removable storage unit to a computer system.

### IX. ADDITIONAL CONSIDERATIONS

Specific details are given in the above description to provide a thorough understanding of the embodiments. However, it is recognized that the embodiments described can be implemented without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above may be accomplished in a variety of ways. For example, these techniques, blocks, steps, and means may be implemented in hardware, software or a combination thereof. For a hardware implementation, the processing units may be implemented within one or more application specifically integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a subsystem, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with subsystems (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium,” “storage,” or “memory” can represent one or more memories for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes, but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

1. A computer-implemented method, comprising:
obtaining a plurality of reference sets of hashed k-mers, wherein each reference set of the plurality of reference sets corresponds to a virus strain of a plurality of subtypes of a virus, wherein each reference set is generated by performing a hash function on k-mers of the virus strain, and wherein each hashed k-mer is stored as a digital key or index to store corresponding k-mer;
obtaining one or more query sequences for a test sample, wherein the query sequence comprises viral nucleotide sequence data derived from the test sample;
generating, for each of the one or more query sequences, a test set of hashed k-mers for the test sample using the hash function, thereby generating one or more test sets;
comparing the one or more test sets to each reference set of the plurality of reference sets to generate a k-mer matching score for each reference set;
determining a query identity for the test sample with respect to each virus strain based on the comparison; and
determining an assigned subtype for the test sample based on the k-mer matching scores and/or the query identities using one or more decision rules.

2. (canceled)

3. The computer-implemented method of claim 1, wherein the k-mer matching score is generated based on a number or percentage of hashed k-mers from the test set that are present in the reference set.

4. The computer-implemented method of claim 1, wherein the obtaining the plurality of reference sets of hashed k-mers comprises:
accessing a virus data store to obtain a plurality of virus strains of the plurality of subtypes of the virus, wherein each virus strain of the plurality of virus strains has a genome sequence and an associated subtype annotation;
generating, for each virus strain of the plurality of virus strains, a set of k-mers by extracting nucleotide sequences of a specific length from the genome sequence using a sliding window; and
performing, for each virus strain of the plurality of virus strains, the hash function on each k-mer of the set of k-mers to generate a digital representation of the hashed k-mer, thereby obtaining the set of hashed k-mers for the virus strain.

5. The computer-implemented method of claim 4, wherein the specific length is 15.

6. The computer-implemented method of claim 4, wherein the performing comprises:
assigning an integer value to each k-mer of the set of k-mers;
combining the integer values based on the hashing function to generate a unique or nearly unique integer for each k-mer; and
assigning each resulting integer as the digital key or index in a hash table or a hash set.

7. The computer-implemented method of claim 1, wherein the generating the test set comprises:
generating a set of k-mers for the test sample by extracting nucleotide sequences of the specific length from the query sequence; and
performing the hashing function on each k-mer of the set of k-mers to generate a digital representation of the hashed k-mer, thereby generating the test set of hashed k-mers.

8. The computer-implemented method of claim 1, wherein the query identity is calculated as a percentage of bases in a genome sequence of the virus strain covered by matching k-mers from the test set.

9. The computer-implemented method of claim 1, wherein the obtaining the one or more query sequences comprises obtaining nucleic acids from the test sample, sequencing the nucleic acids using a high-throughput sequencing technique or capillary electrophoresis sequencing, and generating raw reads or consensus sequences.

10. (canceled)

11. (canceled)

12. (canceled)

13. The computer-implemented method of claim 1, wherein the determining the assigned subtype comprises:
filtering virus strains with a query identity below a predetermined threshold; and
determining the assigned subtype based on remaining virus strains, wherein when candidate strains meeting a predetermined criterion of the query identity share a same subtype, assigning the subtype to the test sample, and when the candidate strains meeting the predetermined criterion of the query identity do not all share the same subtype, applying a weighted voting rule to assign a first subtype and a second subtype based on the weighted voting rule, wherein the first subtype is assigned a higher confidence score than a confidence score of the second subtype.

14. (canceled)

15. The computer-implemented method of claim 1, wherein the virus is human immunodeficiency virus (HIV), hepatitis B virus (HBV), hepatitis C virus (HCV), or hepatitis delta virus (HDV).

16. The computer-implemented method of claim 1, wherein the determining the query identity comprises:
identifying a subset of k-mers from the test sample that have matching hashed values in the reference set for the virus strain;
generating a consensus sequence for the test sample with respect to the virus strain based on the subset of k-mers; and
calculating the query identity as a percentage of bases in a genome sequence of the virus strain that are covered by the consensus sequence.

17. (canceled)

18. The computer-implemented method of claim 1, wherein the assigned subtype for the test sample is (i) a subtype from the plurality of subtypes or (ii) marked as “undetermined.”

19. A non-transitory computer-readable medium comprising computer program instruction that, when executed by one or more processors, cause the one or more processors to perform operations comprising:
obtaining a plurality of reference sets of hashed k-mers, wherein each reference set of the plurality of reference sets corresponds to a virus strain of a plurality of subtypes of a virus, wherein each reference set is generated by performing a hash function on k-mers of the virus strain, and wherein each hashed k-mer is stored as a digital key or index to store corresponding k-mer;
obtaining one or more query sequences for a test sample, wherein the query sequence comprises viral nucleotide sequence data derived from the test sample;
generating, for each of the one or more query sequences, a test set of hashed k-mers for the test sample using the hash function, thereby generating one or more test sets;
comparing the one or more test sets to each reference set of the plurality of reference sets to generate a k-mer matching score for each reference set;
determining a query identity for the test sample with respect to each virus strain based on the comparison; and
determining an assigned subtype for the test sample based on the k-mer matching scores and/or the query identities using one or more decision rules.

20. A system, comprising:
one or more processors; and
a non-transitory computer readable medium containing instructions which, when executed on the one or more processors, cause the one or more processors to perform operations comprising:
obtaining a plurality of reference sets of hashed k-mers, wherein each reference set of the plurality of reference sets corresponds to a virus strain of a plurality of subtypes of a virus, wherein each reference set is generated by performing a hash function on k-mers of the virus strain, and wherein each hashed k-mer is stored as a digital key or index to store corresponding k-mer;
obtaining one or more query sequences for a test sample, wherein the query sequence comprises viral nucleotide sequence data derived from the test sample;
generating, for each of the one or more query sequences, a test set of hashed k-mers for the test sample using the hash function, thereby generating one or more test sets;
comparing the one or more test sets to each reference set of the plurality of reference sets to generate a k-mer matching score for each reference set;
determining a query identity for the test sample with respect to each virus strain based on the comparison; and
determining an assigned subtype for the test sample based on the k-mer matching scores and/or the query identities using one or more decision rules.

21. The non-transitory computer-readable medium of claim 19, wherein the obtaining the plurality of reference sets of hashed k-mers comprises:
accessing a virus data store to obtain a plurality of virus strains of the plurality of subtypes of the virus, wherein each virus strain of the plurality of virus strains has a genome sequence and an associated subtype annotation;
generating, for each virus strain of the plurality of virus strains, a set of k-mers by extracting nucleotide sequences of a specific length from the genome sequence using a sliding window; and
performing, for each virus strain of the plurality of virus strains, the hash function on each k-mer of the set of k-mers to generate a digital representation of the hashed k-mer, thereby obtaining the set of hashed k-mers for the virus strain.

22. The system of claim 20, wherein the obtaining the plurality of reference sets of hashed k-mers comprises:
accessing a virus data store to obtain a plurality of virus strains of the plurality of subtypes of the virus, wherein each virus strain of the plurality of virus strains has a genome sequence and an associated subtype annotation;
generating, for each virus strain of the plurality of virus strains, a set of k-mers by extracting nucleotide sequences of a specific length from the genome sequence using a sliding window; and
performing, for each virus strain of the plurality of virus strains, the hash function on each k-mer of the set of k-mers to generate a digital representation of the hashed k-mer, thereby obtaining the set of hashed k-mers for the virus strain.

23. The non-transitory computer-readable medium of claim 19, wherein the determining the assigned subtype comprises:
filtering virus strains with a query identity below a predetermined threshold; and
determining the assigned subtype based on remaining virus strains, wherein when candidate strains meeting a predetermined criterion of the query identity share a same subtype, assigning the subtype to the test sample, and when the candidate strains meeting the predetermined criterion of the query identity do not all share the same subtype, applying a weighted voting rule to assign a first subtype and a second subtype based on the weighted voting rule, wherein the first subtype is assigned a higher confidence score than a confidence score of the second subtype.

24. The system of claim 20, wherein the determining the assigned subtype comprises:
filtering virus strains with a query identity below a predetermined threshold; and
determining the assigned subtype based on remaining virus strains, wherein when candidate strains meeting a predetermined criterion of the query identity share a same subtype, assigning the subtype to the test sample, and when the candidate strains meeting the predetermined criterion of the query identity do not all share the same subtype, applying a weighted voting rule to assign a first subtype and a second subtype based on the weighted voting rule, wherein the first subtype is assigned a higher confidence score than a confidence score of the second subtype.

25. The non-transitory computer-readable medium of claim 19, wherein the determining the query identity comprises:
identifying a subset of k-mers from the test sample that have matching hashed values in the reference set for the virus strain;
generating a consensus sequence for the test sample with respect to the virus strain based on the subset of k-mers; and
calculating the query identity as a percentage of bases in a genome sequence of the virus strain that are covered by the consensus sequence.

26. The system of claim 20, wherein the determining the query identity comprises:
identifying a subset of k-mers from the test sample that have matching hashed values in the reference set for the virus strain;
generating a consensus sequence for the test sample with respect to the virus strain based on the subset of k-mers; and
calculating the query identity as a percentage of bases in a genome sequence of the virus strain that are covered by the consensus sequence.
